SEQUENCE LISTING (3 sequences)

SEQ ID NO: 1 - DNA, 12 bp, Escherichia coli
tcccctcctttt

SEQ ID NO: 2 - DNA, 12 bp, Artificial Sequence
  Description of Artificial Sequence Synthetic oligonucleotide
aaaaggagggga

SEQ ID NO: 3 - protein, 6 aa, Artificial Sequence
  Description of Artificial Sequence Synthetic 6xHis tag
HHHHHH

(19) United States
(12) Patent Application Publication
(10) Pub. No.: US 2019/0055592 A1
(43) Pub. Date: Feb. 21, 2019

(54) Nanopore Discrimination of Target Polynucleotides from Sample Background by Fragmentation and Payload Binding

(71) Applicant: Two Pore Guys, Inc., Santa Cruz, CA (US)

(72) Inventors: Trevor J. Morin, Santa Cruz, CA (US); William B. Dunbar, Santa Cruz, CA (US); Daniel A. Heller, Santa Cruz, CA (US); Tyler Shropshire, Santa Cruz, CA (US)

(21) Appl. No.: 16/079,762

(22) PCT Filed: Mar. 31, 2017
(86) PCT No.: PCT/US2017/025585

§ 371 Date: Aug. 24, 2018

Related U.S. Application Data
(60) Provisional application No. 62/316,452, filed on Mar. 31, 2016.
(60) Provisional application No. 62/354,068, filed on Jun. 23, 2016.
(60) Provisional application No. 62/354,068, filed on Jun. 23, 2016.
(60) Provisional application No. 62/412,221, filed on Oct. 24, 2016.

(51) Int. Cl. C12Q 1/6825; G01N 33/487; G01N 27/447

(52) U.S. Cl. CPC C12Q 1/6825; G01N 27/44791; G01N 33/48721

(57) ABSTRACT

Disclosed herein are methods and compositions for detecting a target DNA sequence from a sample that does not require sample purification or amplification. The method uses fragmentation, sequence-specific binding or ligation of probes, and payload molecules for selective detection of the target-sequence using a nanopore sensor.

### CROSS-REFERENCE TO RELATED APPLICATIONS

This application claims the benefit under 35 U.S.C § 119(e) to U.S. Provisional Application No. 62/316,452, filed Mar. 31, 2016, U.S. Provisional Application No. 62/354,068, filed Jun. 23, 2016, and U.S. Provisional Application No. 62/412,221, filed Oct. 24, 2016, the contents of each of which are incorporated by reference in their entirety.

### FIELD OF THE INVENTION

The invention relates to methods and compositions for target sequence detection using a nanopore device.

### BACKGROUND

Detecting nucleic acid specific to an organism is an accurate and efficient method for identifying microbes, viruses, and other infection agents. Additionally, detecting a specific nucleic acid sequence, or detecting the presence or absence of a segment of DNA comprising a specific sequence, can identify disease-causing mutations. Being able to accomplish this has applications in biomedical science and technology, medicine, agriculture and forensics, as well as in other fields.

The detection of genes and their modifications, sequence, location, or number, is important for the advancement of molecular diagnostics in medicine. DNA microarrays, PCR, Southern Blots, and FISH (Fluorescent in situ Hybridization) are all methods that can be used to perform or aid nucleic acid detection. These methods can be slow and labor intensive, and have limited accuracy and resolution. More recent methods, such as real-time PCR and next-generation sequencing (NGS) technologies, have improved throughput and accuracy, but require complex and costly device infrastructure to perform quantitation, and typically incorporate some form of optics for sensing.

By comparison, a solid state nanopore can provide a nucleic acid sensor that is electrical, without the need for optics. Moreover, solid-state nanopore devices can be made using scalable fabrication techniques at very low cost, and incorporated into small form factors for portable use.

Solid-state nanopores can detect molecules by applying a voltage across the pore, and measuring current impedance changes (“events”) as the molecules pass through the nanopore. The overall efficacy of any given nanopore device depends on its ability to accurately and reliably measure impedance events above noise, and to discriminate events that are due to molecules of interest from events due to any background molecules when present.

Experiments published in the literature have demonstrated both the detection of purified DNA and RNA strands passing through the pores. DNA with synthetic molecules bound to specific sequences has been shown to permit target sequence detection, but only I the context of a purified DNA sample with defined polynucleotide lengths. In practical applications of detecting DNA from a sample, polynucleotides can exceed 1 Mbp in length, and can clog or prevent detection in a nanopore.

Thus, what is needed are methods to facilitate target sequence detection in a nanopore that is tolerant to background molecules and can handle samples with very long DNA molecules.

### SUMMARY

In some embodiments, provided herein is a method of detecting the presence or absence of a target polynucleotide sequence suspected to be present in a sample, the method comprising: providing a sample suspected of containing a polynucleotide comprising a target sequence; fragmenting said polynucleotide; providing a probe adapted to bind specifically to said target sequence of said polynucleotide; contacting said sample with said probe under conditions that promote binding of said probe to said target sequence to form a polynucleotide-probe complex; loading said sample into a nanopore device comprising a nanopore, a first chamber, and a second chamber, wherein said first and second chamber are in electrical and fluidic communication through said nanopore via a conducting fluid, and wherein said nanopore device further comprises a sensor configured to identify objects passing through the nanopore; applying an electrical potential across said nanopore to induce translocation of said polynucleotide or polynucleotide-probe complex through said nanopore; and detecting an electrical signal associated with the translocation of said polynucleotide or polynucleotide-probe complex through the nanopore. In some embodiments, the method further comprises analyzing said electrical signal to determine the presence or absence of said target polynucleotide sequence in said sample. In some embodiments, the probe is bound to a payload molecule.

In some embodiments, the probe comprises a payload binding moiety. In some embodiments, the payload binding moiety comprises a chemical group, a reactive group, a small molecule, or a peptide. In some embodiments, the small molecule comprises biotin. In some embodiments, the reactive group comprises dibenzocyclooctyl (DBCO) or azide. In some embodiments, the reactive group comprises a reactive maleimide, a free thiol (thiolate), or a sulfur atom.

In some embodiments, the method further comprises binding a payload molecule to said payload binding moiety before applying said electrical potential. In some embodiments, the payload molecule is bound to said payload binding moiety after contacting said sample with said probe. In some embodiments, the payload molecule is bound to said payload binding moiety before contacting said sample with said probe.

In some embodiments, the payload molecule is selected from the group consisting of: a dendrimer, double stranded DNA, single stranded DNA, a DNA aptamer, a fluorophore, a protein, an antibody, a polypeptide, a nanobead, a nanorod, a nanotube, nanoparticle, fullerene, a PEG molecule, a liposome, or a cholesterol-DNA hybrid.

In some embodiments, the payload molecule comprises an electrical charge. In some embodiments, the charged payload molecule is selected from the group consisting of: a peptide, an amino acid, a charged nanoparticle, a synthetic molecule, a nucleotide, a polynucleotide, a metal, and an ion. In some embodiments, the sensitivity or specificity of detection of the presence of absence of the target polynucleotide is increased when said target polynucleotide is bound to said charged payload molecule as compared to unbound target polynucleotide.

In some embodiments, the payload binding moiety and the payload molecule are bound via a covalent bond. In some embodiments, the covalent bond is formed by click chemistry. In some embodiments, the click chemistry is copper catalyzed. In some embodiments, the click chemistry is copper free. In some embodiments, the covalent bond comprises a thio-ether bond. In some embodiments, the thio-ether bond is formed by maleimido-thiolate chemistry.

In some embodiments, the payload binding moiety and the payload molecule are bound via a non-covalent bond. In some embodiments, the non-covalent bond is selected from the group consisting of: a hydrogen bond, an ionic bond, a van der Waals interaction, a hydrophobic interaction, a polar bond, a cation-pi interaction, a planar stacking interaction, and a metallic bond.

In some embodiments, the sensitivity or specificity of detection of the presence or absence of the target polynucleotide is increased when said target polynucleotide is bound to said payload molecule as compared to unbound target polynucleotide.

In some embodiments, two or more payload molecules are bound to the target polynucleotide.

In some embodiments, the specific binding of said probe to said target sequence of said polynucleotide occurs via sequence-specific ligation.

In some embodiments, fragmenting said polynucleotide comprises exposing said sample to a fragmentation condition. In some embodiments, the fragmentation condition is selected from the group consisting of: chemical shearing, heat and divalent metal cation, acoustic shearing, sonication, hydrodynamic shearing, nebulization, needle shearing, and French pressing. In some embodiments, the fragmenting said polynucleotide comprises contacting said sample with a fragmentation reagent. In some embodiments, the fragmentation reagent is selected from the group consisting of: a restriction enzyme, a site-directed nuclease, endonuclease, non-specific nuclease, transposase, and catalytic DNA or RNA.

In some embodiments, the sample comprises a plurality of target polynucleotides. In some embodiments, providing said probe comprises providing a plurality of unique probes adapted to specifically bind to target sequence so that each of said plurality of target polynucleotide-probe complexes generates a unique and detectable signal upon translocation through the nanopore. In some embodiments, contacting said sample with said probe comprises contacting said sample with said plurality of unique probes. In some embodiments, the method comprises detecting an electrical signal associated with the translocation of at least one of said plurality of target polynucleotide-probe complexes.

In some embodiments, the nanopore device comprises at least two nanopores, and wherein said nanopore device is configured to apply an independently-controlled voltage across each of said at least two nanopores. In some embodiments, the at least two nanopores are in series. In some embodiments, the method further comprises capturing a polynucleotide or polynucleotide-probe complex in at least two nanopores in said device simultaneously.

In some embodiments, the sample is loaded into said device before said fragmentation of said polynucleotide. In some embodiments, the sample is loaded into said device after said fragmentation of said polynucleotide. In some embodiments, the sample is loaded into said device before said contacting of said sample with said probe. In some embodiments, the sample is loaded into said device after said contacting of said sample with said probe.

In some embodiments, the sample is not purified. In some embodiments, the sample is not purified before said fragmentation, before contacting said sample with said probe, or before said detection in said nanopore. In some embodiments, the sample is loaded into said nanopore device at a dilution of at least 1:20000, 1:10000, 1:5000, 1:2000, 1:1000, 1:500, 1:200, 1:100, 1:50, 1:20, 1:10, 1:5, 1:2, 1:1.5, 1:1.2, 1:1.1 or 1:1.05. In some embodiments, the sample is loaded into said nanopore device without dilution. In some embodiments, the sample comprises non-target polynucleotides, fragmentation reaction reagents, and ligation reaction reagents while in said nanopore device.

In some embodiments, the nanopore is at least 5 nm, 10 nm, 20 nm, 20 nm, 40 nm, or 50 nm in diameter. In some embodiments, the nanopore is less than 200 nm in diameter.

In some embodiments, fragmenting said polynucleotide comprises a sequence-specific fragmentation reaction. In some embodiments, the sequence-specific fragmentation reaction comprises site-specific restriction enzymes or CRISPR-based cleavage. In some embodiments, fragmenting said polynucleotide comprises a non-sequence-specific fragmentation reaction. In some embodiments, the non-sequence-specific fragmentation reaction is achieved by shearing.

In some embodiments, the probe is contacted with said sample in the interior space of the nanopore device.

In some embodiments, the target polynucleotide comprises double-stranded deoxyribonucleic acid (dsDNA), single-stranded DNA (ssDNA), peptide nucleic acid (PNA), single-stranded ribonucleic acid (ssRNA), DNA/RNA hybrid, or double-stranded ribonucleic acid (dsRNA). In some embodiments, the target polynucleotide is a naturally-occurring polynucleotide. In some embodiments, the target polynucleotide is an artificially-synthesized polynucleotide. In some embodiments, the target polynucleotide is a recombinant polynucleotide.

In some embodiments, the sensor comprises an electrode pair configured to generate said electrical potential across said nanopore and to detect said electrical signal. In some embodiments, the electrical signal generated when the payload-bound target polynucleotide passes through the nanopore is distinguishable from the electrical signal of background molecules. In some embodiments, the electrical signal is a measure of current over time, and the electrical signal is distinguishable by its mean depth, maximum depth, duration, number of depth levels, area of depth and duration, or noise level.

Also provided herein is a method of quantifying a target polynucleotide sequence in a sample, the method comprising: providing a sample suspected of containing a polynucleotide comprising a target sequence; fragmenting said polynucleotide; providing a probe adapted to bind specifically to said target sequence of said polynucleotide; contacting said sample with said probe under conditions that promote binding of said probe to said target sequence to form a polynucleotide-probe complex; loading said sample into a nanopore device comprising a nanopore, a first chamber, and a second chamber, wherein said first and second chamber are in electrical and fluidic communication through said nanopore via a conducting fluid, and wherein said nanopore device further comprises a sensor configured to identify objects passing through the nanopore; applying an electrical potential across said nanopore to induce translocation of said polynucleotide or polynucleotide-probe complex through said nanopore; detecting an electrical signal associated with the translocation of said polynucleotide or polynucleotide-probe complex through the nanopore; and analyzing said electrical signal to determine a measurement of quantity of said target polynucleotide sequence in said sample.

In some embodiments, the probe is bound to a payload molecule. In some embodiments, the probe comprises a payload binding moiety. In some embodiments, the payload molecule is bound to said payload binding moiety after contacting said sample with said probe.

Also provided herein is a kit, the kit comprising: a device comprising a nanopore, wherein said nanopore separates an interior space of the device into two volumes, wherein the device comprises a sensor for said nanopore adapted to identify objects passing through the nanopore; a probe adapted to bind specifically to a target sequence of a polynucleotide; and instructions for use to detect the presence or absence of said target sequence in a sample.

In some embodiments, the probe is bound to a payload molecule. In some embodiments, the probe comprises a payload binding moiety. In some embodiments, the kit comprises a payload molecule adapted to bind to said payload binding moiety. In some embodiments, the kit comprises reagents for fragmenting said polynucleotide.

Provided herein are methods of detecting a polynucleotide comprising a target sequence in a sample, comprising: contacting said sample with a probe that specifically binds to said polynucleotide comprising said target sequence under conditions that promote binding of said probe to said target sequence to form a polynucleotide-probe complex; loading said sample into a first chamber of a nanopore device, wherein said nanopore device comprises at least one nanopore and at least said first chamber and a second chamber, wherein said first and second chamber are in electrical and fluidic communication through said at least one nanopore, and wherein the nanopore device further comprises an independently-controlled voltage across each of said at least one nanopores and a sensor associated with each of said at least one nanopores, wherein said sensor is configured to identify objects passing through the at least one nanopore, and wherein said polynucleotide-probe complex translocating through said at least one nanopore provides a detectable signal associated with said polynucleotide-probe complex; and determining the presence or absence of said polynucleotide-probe complex in said sample by observing said detectable signal, thereby detecting said polynucleotide comprising said target sequence. In an embodiment, the method further comprises generating a voltage potential through said at least one nanopore, wherein said voltage potential generates a force on said polynucleotide-probe complex to pull said polynucleotide-probe complex through said at least one nanopore, causing said polynucleotide-probe complex to translocate through said at least one nanopore to generate said detectable signal.

In some embodiments, said polynucleotide is DNA or RNA. In an embodiment, said detectable signal is an electrical signal. In an embodiment, said detectable signal is an optical signal. In an embodiment, said probe comprises a molecule selected from the group consisting of: a protein, a peptide, a nucleic acid, a TALEN, a CRISPR, a peptide nucleic acid, or a chemical compound. In an embodiment, said probe comprises a molecule selected from the group consisting of: a deoxyribonucleic acid (DNA), a ribonucleic acid (RNA), a peptide nucleic acid (PNA), a DNA/RNA hybrid, polypeptide, or any chemically derived polymer.

In an embodiment, said probe comprises a PNA molecule bound to a secondary molecule configured to facilitate detection of the probe bound to said polynucleotide during translocation through said at least one nanopore. In a further embodiment, said secondary molecule is a PEG. In a further embodiment, said PEG has a molecular weight of at least 1 kDa, 2 kDa, 3 kDa, 4 kDa, 5 kDa, 6 kDa, 7 kDa, 8 kDa, 9 kDa, or 10 kDa.

In an embodiment, said method of detecting a polynucleotide comprising a target sequence in a sample further comprises applying a condition to said sample suspected to alter the binding interaction between the probe and the target sequence. In a further embodiment, the condition is selected from the group consisting of: removing the probe from the sample, adding an agent that competes with the probe for binding to the target sequence, and changing an initial pH, salt, or temperature condition.

In an embodiment, said polynucleotide comprises a chemical modification configured to modify binding of the polynucleotide to the probe. In a further embodiment, the chemical modification is selected from the group consisting of biotinylation, acetylation, methylation, summolation, glycosylation, phosphorylation and oxidation.

In an embodiment, said probe comprises a chemical modification coupled to the probe through a cleavable bond. In an embodiment, said probe interacts with the target sequence of the polynucleotide via a covalent bond, a hydrogen bond, an ionic bond, a metallic bond, van der Waals force, hydrophobic interaction, or planar stacking interactions. In an embodiment, said method of detecting a polynucleotide comprising a target sequence in a sample further comprises contacting the sample with one or more detectable labels capable of binding to the probe or to the polynucleotide-probe complex. In an embodiment, said polynucleotide comprises at least two target sequences.

In an embodiment, said nanopore is about 1 nm to about 100 nm in diameter, 1 nm to about 100 nm in length, and wherein each of the chambers comprises an electrode. In an embodiment, said nanopore device comprises at least two nanopores configured to control the movement of said polynucleotide in both nanopores simultaneously. In an embodiment, said method of detecting a polynucleotide comprising a target sequence in a sample further comprises reversing said independently-controlled voltage after initial detection of the polynucleotide-probe complex by said detectable signal, so that the movement of said polynucleotide through the nanopore is reversed after the probe-bound portion passes through the nanopore, thereby identifying again the presence or absence of a polynucleotide-probe complex.

In an embodiment, said nanopore device comprises two nanopores, and wherein said polynucleotide is simultaneously located within both of said two nanopores. In a further embodiment, said method of detecting a polynucleotide comprising a target sequence in a sample comprises comprising adjusting the magnitude and or the direction of the voltage in each of said two nanopores so that an opposing force is generated by the nanopores to control the rate of translocation of the polynucleotide through the nanopores.

Also provided herein is a method of detecting a polynucleotide or a polynucleotide sequence in a sample, comprising: contacting said sample with a first probe and a second probe, wherein said first probe specifically binds to a first target sequence of said polynucleotide under conditions that promote binding of said first probe to said first target sequence, wherein said second probe specifically binds to a second target sequence of said polynucleotide under conditions that promote binding of said second probe to said second target sequence; contacting said sample with a third molecule is configured to bind to said first and second probe simultaneously when said first and second probe are within a sufficient proximity to each other under conditions that promote binding of said third molecule to said first probe and said second probe, thereby forming a fusion complex comprising said polynucleotide, said first probe, said second probe, and said third molecule; loading said sample into a first chamber of a nanopore device, wherein said nanopore device comprises at least one nanopore and at least said first chamber and a second chamber, wherein said first and second chamber are in electrical and fluidic communication through said at least one nanopore, and wherein the nanopore device further comprises a controlled voltage potential across each of said at least one nanopores and a sensor associated with each of said at least one nanopores, wherein said sensor is configured to identify objects passing through the at least one nanopore, and wherein said fusion complex translocating through said at least one nanopore provides a detectable signal associated with said fusion complex; and determining the presence or absence of said fusion complex in said sample by observing said detectable signal.

In an embodiment, said polynucleotide is DNA or RNA. In an embodiment, said detectable signal is an electric signal. In an embodiment, said detectable signal is an optical signal. In an embodiment, said sufficient proximity is less than 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 300, 400, or 500 nucleotides. In an embodiment, said third molecule comprises a PEG or an antibody.

In an embodiment, said third molecule and said first and second probes are bound to ssDNA, and wherein said ssDNA linked to said third molecule comprises a region complementary to a region of ssDNA linked to said first probe and is complementary to a region of ssDNA linked to said second probe. In an embodiment, the method of detecting a polynucleotide or a polynucleotide sequence in a sample further comprising contacting the sample with one or more detectable labels capable of binding to the third molecule or to the fusion complex.

Also provided herein is a kit comprising a first probe, a second probe, and a third molecule, wherein the first probe is configured to bind to a first target sequence on a target polynucleotide, wherein the second probe is configured to bind to a second target sequence on said target polynucleotide, and wherein said third molecule is configured to bind to the first probe and the second probe when said first and second probes are bound to said polynucleotide at said first and second target sequences, thereby locating the first and second probe in sufficient proximity to allow binding of said third molecule to said first and second probes simultaneously.

In an embodiment, said first probe and said second probe are selected from the group consisting of: a protein, a peptide, a nucleic acid, a TALEN, a CRISPR, a peptide nucleic acid, or a chemical compound. In an embodiment, said third molecule comprises a PEG or an antibody. In an embodiment, said third molecule comprises a modification to modify binding affinity to said probes.

Also provided herein is a nanopore device comprising at least two chambers and a nanopore, wherein said device comprises a modified PNA probe bound to a polynucleotide within said nanopore.

Also provided herein is a dual-pore, dual-amplifier device for detecting a charged polymer through two pores, the device comprising an upper chamber, a middle chamber and a lower chamber, a first pore connecting the upper chamber and the middle chamber, and a second pore connecting the middle chamber and the lower chamber, wherein said device comprises a modified PNA probe bound to a polynucleotide within said first or second pore.

In an embodiment, the device is configured to control the movement of said charged polymer through both said first pore and said second pore simultaneously. In an embodiment, the modified PNA probe is bound to at least one PEG molecule. In an embodiment, the device further comprises a power supply configured to provide a first voltage between the upper chamber and the middle chamber, and provide a second voltage between the middle chamber and the lower chamber, each voltage being independently adjustable, wherein the middle chamber is connected to a common ground relative to the two voltages, wherein the device provides dual-amplifier electronics configured for independent voltage control and current measurement at each pore, wherein the two voltages may be different in magnitude, wherein the first and second pores are configured so that the charged polymer is capable of simultaneously moving across both pores in either direction and in a controlled manner.

### BRIEF DESCRIPTION OF THE DRAWINGS

Provided as embodiments of this disclosure are drawings which illustrate by exemplification only, and not limitation.

FIG. 1 depicts a polynucleotide comprising a target polynucleotide sequence bound to a payload molecule through the probe, and the complex passing through the nanopore.

FIG. 2 depicts differences in current signatures when a payload-bound target polynucleotide passes through the pore, compared to a non-target background polynucleotide and a generic non-polynucleotide background molecule.

FIG. 3 depicts a method of detecting target sequences from a sample without amplification. In particular, FIG. 3 shows a method to detect a target sequence that involves using site-specific cleavage of the target sequence, and ligating probes that are competent for attaching payload molecules that facilitate nanopore detection.

FIG. 4 illustrates probes of differing size or charge or other configuration to generate a unique signature upon nanopore translocation that each bind to a unique target sequence in the target-bearing molecule.

FIG. 5A shows a PNA ligand that has been modified as to increase ligand charge, and therefore facilitate detection by a nanopore. FIG. 5B shows an example in which a double-stranded DNA is used as the target bearing polymer and multiple different DNA binding probes that bind to target sequences that are desired to be detected.

FIG. 6A shows a PNA-PEG probe bound to its target sequence on a dsDNA molecule. FIG. 6B shows the results of a gel shift assay with the following samples: DNA only (lane 1), DNA/PNA (lane 2), DNA/PNA-PEG (10 kDa) (lane 3), and DNA/PNA-PEG (20 kDa) (lane 4). FIG. 6C shows the results of a gel shift assay with the following samples: DNA marker (lane 1), random DNA sequence incubated with PNA probe (lane 2), DNA with single mismatch at target sequence incubated with corresponding PNA probe (lane 3), and DNA with target sequence mixed with corresponding PNA probe specific to the target sequence (lane 4).

FIG. 7A shows representative current signature events as the molecule depicted below each current signature passes through the nanopore under an applied voltage. FIG. 7B shows a scatter plot of events characterized by duration and mean conductance shift due to translocation through the nanopore in three populations: DNA/bisPNA (square), DNA/bisPNA-PEG 5 kDa (circle), and DNA/bisPNA-PEG 10 kDa (diamond). FIG. 7C shows a histogram of mean conductance shift probability associated with each of the three populations described above. FIG. 7D shows a histogram of event duration probability associated with each of the three populations described above.

FIG. 8A shows representative event signatures correlated with the translocation of a PNA-PEG probe bound to a DNA molecule. FIG. 8B shows the mean conductance shift v. duration plot for each recorded event in the nanopore from a sample comprising bacterial DNA and PNA-PEG probe. FIG. 8C and FIG. 8D show corresponding histograms to characterize these events detected by mean conductance shift and duration of each event respectively. FIG. 8E shows the results of a gel shift assay showing: 100 bp ladder (lane 1), 300 bp DNA with wild type cftr sequence incubated with the PNA-PEG probe (lane 2), and 300 bp DNA with the cftr ΔF508 sequence incubated with the PNA-PEG probe (lane 3).

FIG. 9A shows the results of the gel shift assay, with lane 1 comprising S. mitis bacterial DNA without a bisPNA-PEG bound, and lane 2 comprising S. mitis DNA with a site-specific bisPNA-PEG bound. FIG. 9B shows a scatter plot of mean conductance shift (dG) on the vertical axis vs. duration on the horizontal axis for all recorded events in the two consecutive experiments. The first sample included bacterial DNA with PEG-modified PNA probes (DNA/bisPNA-PEG). The second sample included bacterial DNA alone.

FIG. 10 illustrates a process of fragmentation and binding of a sequence-specific probe comprising a payload to a target sequence, according to an embodiment of the invention.

FIG. 11 is an agarose gel that shows bacterial plasmid fractionation.

FIG. 12 illustrates an exemplary bisPNA probe comprising a region that binds to a specific 12-mer target oligonucleotide sequence, and a cysteine linker capable of forming a covalent bond with a 40 kDa, 3-arm maleimido-PEG payload. FIG. 12 also illustrates an embodiment of the bisPNA probe covalently attached to the 3-arm maleimido-PEG payload and bound to its target DNA sequence.

FIG. 13 shows the results of HPLC purification of bisPNA-PEG conjugation reaction.

FIG. 14 shows the results of detection in the nanopore of the following samples: i) fragmented DNA only, ii) PNA-PEG probe only, iii) DNA mixed with PNA probe, and iv) DNA mixed with DNA probe bound to a payload (4-arm PEG). Panel a) shows the separation of each population on a plot of event duration and maximum δG for each event. Panel b) and c) show probability histograms for values of maximum δG (panel b)) and event duration (panel c)) for each population detected in a nanopore.

FIG. 15 shows an event plot of event duration vs maximum δG for two molecule types (96 bp DNA/probe-payload complex and secondary molecule) that were run sequentially on the same pore.

FIG. 16 illustrates differentiation of the target DNA/probe-payload complex and the secondary molecule and methods to quantify relative abundance of the target to the known amount of secondary molecule.

Some or all of the figures are schematic representations for exemplification; hence, they do not necessarily depict the actual relative sizes or locations of the elements shown. The figures are presented for the purpose of illustrating one or more embodiments with the explicit understanding that they do not limit the scope or the meaning of the claims that follow below.

### DETAILED DESCRIPTION

Throughout this application, the text refers to various embodiments of the present nutrients, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

Also throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

As used in the specification and claims, the singular form “a,” “an” and “the” include plural references unless the context clearly dictates otherwise. For example, the term “an electrode” includes a plurality of electrodes, including mixtures thereof.

As used herein, the term “comprising” is intended to mean that the devices and methods include the recited components or steps, but not excluding others. “Consisting essentially of” when used to define devices and methods, shall mean excluding other components or steps of any essential significance to the combination. “Consisting of” shall mean excluding other components or steps. Embodiments defined by each of these transition terms are within the scope of this invention.

All numerical designations, e.g., distance, size, temperature, time, voltage and concentration, including ranges, are approximations which are intended to encompass ordinary experimental variation in measurement of the parameters, and that variations are intended to be within the scope of the described embodiment. It is to be understood, although not always explicitly stated that all numerical designations are preceded by the term “about”. It also is to be understood, although not always explicitly stated, that the components described herein are merely exemplary and that equivalents of such are known in the art.

As used herein, the term “target sequence” refers to a portion of a polynucleotide having a sequence of nucleic acids of interest. The target sequence can be specifically targeted by reagents for separating (i.e., fragmenting) a polynucleotide into a plurality of fragmented segments. The target sequence can also be specifically targeted for binding by a probe to facilitate detection of the target sequence in a nanopore sensor, as described herein.

As used herein, the term “fragmenting” refers to a physical separation of a polynucleotide into at least two polynucleotide fragments. This can be accomplished by exposing the polynucleotide to conditions that facilitate separation of the polynucleotide. This can also be accomplished by exposing the polynucleotide to an enzyme or other reagent that facilitates separation of a polynucleotide into two or more fragments. This fragmentation can be designed to occur at specific target sequences on a polynucleotide.

As used herein, the term “ligation” refers to binding of a probe to a polynucleotide comprising a target sequence. In some embodiments, the polynucleotide comprising the target sequence has been fragmented. As an example, ligation of the probe to the polynucleotide can occur through binding via a complementary sequence, or can be facilitated by a ligation enzyme.

As used herein, the term “specific binding” or “bind specifically” refers to the targeted binding of a probe to a polynucleotide comprising a target sequence or to a fragment thereof.

As used herein, the term “probe” refers to a molecule that binds specifically to a polynucleotide comprising a target sequence or to a fragment thereof. In some embodiments, the probe comprises a payload molecule. In some embodiments, the probe comprises a payload molecule binding moiety adapted to bind to a payload molecule.

As used herein, the term “payload molecule” refers to a molecule with physical dimensions that facilitate generation of a unique electrical signal when captured in a nanopore within a correlated range of dimensions. A payload molecule may be attached to a target molecule to facilitate detection of the target molecule in a nanopore device. In some embodiments, the payload molecule may also be charged to act as a driver molecule. In some embodiments, the payload molecule comprises a probe binding moiety capable of specifically binding a probe molecule.

The term “nanopore” (or, just “pore”) as used herein refers to a single nano-scale opening in a membrane that separates two volumes. The pore can be a protein channel inserted in a lipid bilayer membrane, for example, or can be engineered by drilling or etching or using a voltage-pulse method through a thin solid-state substrate, such as silicon nitride or silicon dioxide or graphene or layers of combinations of these or other materials. Geometrically, the pore has dimensions no smaller than 0.1 nm in diameter and no bigger than 1 micron in diameter; the length of the pore is governed by the membrane thickness, which can be sub-nanometer thickness, or up to 1 micron or more in thickness. For membranes thicker than a few hundred nanometers, the nanopore may be referred to as a “nano channel.”

As used here, the term “nanopore instrument” or “nanopore device” refers to a device that combines one or more nanopores (in parallel or in series) with circuitry for sensing single molecule events. Specifically, nanopore instruments use a sensitive voltage-clamp amplifier to apply a specified voltage across the pore or pores while measuring the ionic current through the pore(s). When a single charged molecule such as a double-stranded DNA (dsDNA) is captured and driven through the pore by electrophoresis, the measured current shifts, indicating a capture event (i.e., the translocation of a molecule through the nanopore, or the capture of a molecule in the nanopore), and the shift amount (in current amplitude) and duration of the event are used to characterize the molecule captured in the nanopore. After recording many events during an experiment, distributions of the events are analyzed to characterize the corresponding molecule according to its shift amount (i.e., its current signature). In this way, nanopores provide a simple, label-free, purely electrical single-molecule method for biomolecular sensing.

As used herein, the term “electrical signal” encompasses a series of data collected on current, impedance/resistance, or voltage over time depending on configuration of the electronic circuitry. Conventionally, current is measured in a “voltage clamp” configuration; voltage is measured in a “current clamp” configuration, and resistance measurements can be derived in either configuration using Ohm's law V=IR. Impedance can also be generated by measured from current or voltage data collected from the nanopore device. Types of electrical signals referenced herein include current signatures and current impedance signatures, although various other electrical signals may be used to detect particles in a nanopore.

As used herein, the term “event” refers to a translocation of a detectable molecule or molecular complex through the nanopore and its associated measurement via an electrical signal, e.g., change in current through the nanopore over time. It can be defined by its current, duration, and/or other characteristics of detection of the molecule in the nanopore. A plurality of events with similar characteristics is indicative of a population of molecules or complexes that are identical or have similar characteristics (e.g., bulk, charge).

As used herein, the term “cleavable linker” or “labile linker” refers to a substrate linker sensitive to enzymatic, photolytic, or chemical cleavage by a target molecule or condition. In some embodiments, the cleavable linker can be a deoxyribonucleic acid (DNA), a polypeptide, a carbon-oxygen bond, a carbon-sulfur bond, a carbon-nitrogen bond, or a carbon-carbon bond. In some embodiments, the cleavable linker sensitive to photolytic cleavage can be an ortho-nitrobenzyl derivative or phenacyl ester derivative. In some embodiments, the cleavable linker sensitive to chemical cleavage can be an azo compounds, disulfide bridge, sulfone, ethylene glycolyl disuccinate, hydrazone, acetal, imine, vinyl ether, vicinal diol, or picolinate ester.

Molecular Detection

The present disclosure provides methods and systems for molecular detection and quantitation. In addition, the methods and systems can also be configured to measure the affinity of a probe binding to a target molecule. Further, such detection, quantitation, and measurement can be carried out in a multiplexed manner, greatly increasing its efficiency.

Thus, provided herein are compositions and methods for detecting or quantifying a polynucleotide that contains a target sequence that is desired to be detected or quantitated.

For nucleic acids and polypeptides to which the target sequence detection method is applied, a target sequence can be a polynucleotide sequence that is recognizable by the probe molecule. Target sequences may be chemically modified (e.g. methylated) or occupied by other molecules (e.g. activator or repressors), and depending on the nature of the probe, the binding status of the target sequence can be elucidated. In some aspects, the target sequence comprises a chemical modification for binding the probe to the polynucleotide. In some aspects, the chemical modification is selected from the group consisting of acetylation, methylation, summolation, glycosylation, phosphorylation, biotinylation, and oxidation.

To facilitate detection of the target sequence using a nanopore device, the target DNA can be fragmented. In some embodiments, fragmentation occurs at sequence-specific locations on the target DNA. In some embodiments, fragmentation generates a set of identical length sequences comprising at least a portion of the DNA. In some embodiments, the target DNA is fragmented at the target sequence of interest. Fragmentation can provide target DNA having uniform lengths to facilitate accurate detection of target DNA by generating more consistent and/or more distinguishable current signatures upon translocation through a nanopore. This fragmentation can be paired with binding or ligation of a probe specific for the DNA comprising at least part of the target sequence to enhance detection in a nanopore.

Thus, also provided herein are probes capable of binding to a specific target sequence on the polynucleotide. These probes can be ligated to the end of fragmented DNA, or can bind to a target sequence on the fragmented DNA The probe can also comprise or be bound to a payload molecule to aid detection of the polynucleotide-probe complex in a nanopore by altering the dwell time or current.

In one embodiment, if all are present in a solution, a probe binds to a target sequence through the specific recognition of the probe for the target sequence. Such binding causes the formation of a complex that includes the probe and the target sequence.

The formed polynucleotide-probe complex can be detected by a nanopore device. The nanopore device includes electronic components to deliver controlled voltages across one or more nanopores (which voltages can, in some embodiments, be independently controlled and clamped) along with circuitry for measuring current flow across the nanopores. An electrical potential, (e.g., a voltage differential) applied across each nanopore facilitates the capture and translocation of a charged polynucleotide through application of an electrostatic force on the charged polynucleotide exposed to the voltage field. Unless specified below, references to a pore or nanopore or nanopore device are intended to encompass single, dual or multi-pore devices within the spirit of the present invention.

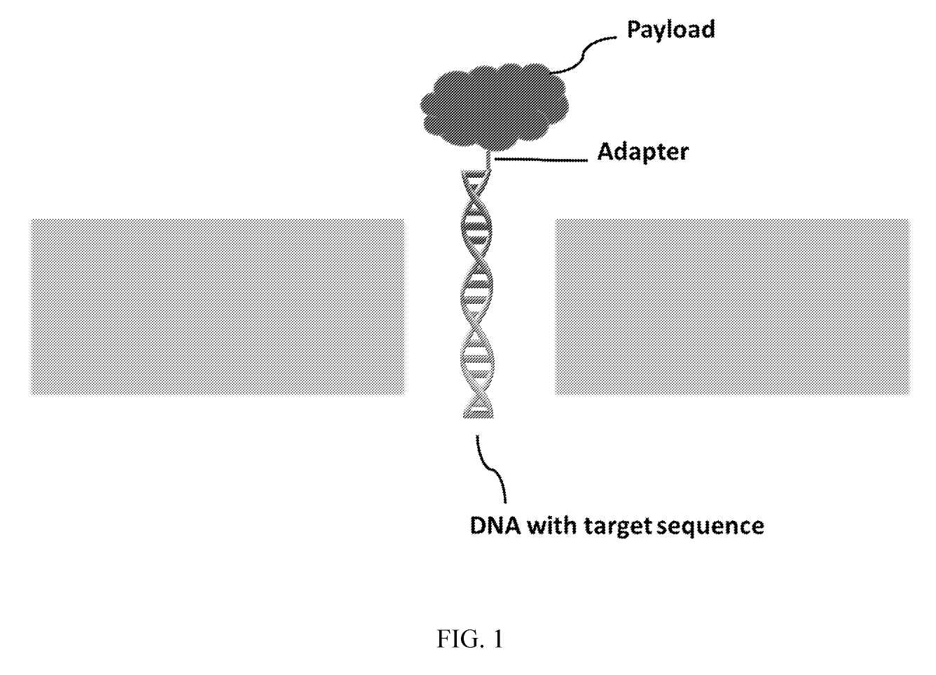

When a sample that includes the polynucleotide target sequence is in the nanopore device, the nanopore can be configured to capture and pass the polynucleotide target sequence through the nanopore. For example, as shown in FIG. 1 a polynucleotide comprising a target sequence is specifically bound by a probe comprising a payload molecule. As shown in FIG. 1, the probe can be bound to the payload molecule through an adapter. In some embodiments, the payload may be bound to the target sequence through ligation after, e.g., enzymatic detection. When the target sequence is within the pore (as shown in FIG. 1) or adjacent to the pore, the binding status of the target sequence can be detected by the sensor, e.g., due to a unique electrical signature generated by the complex's measured effect on current through the pore.

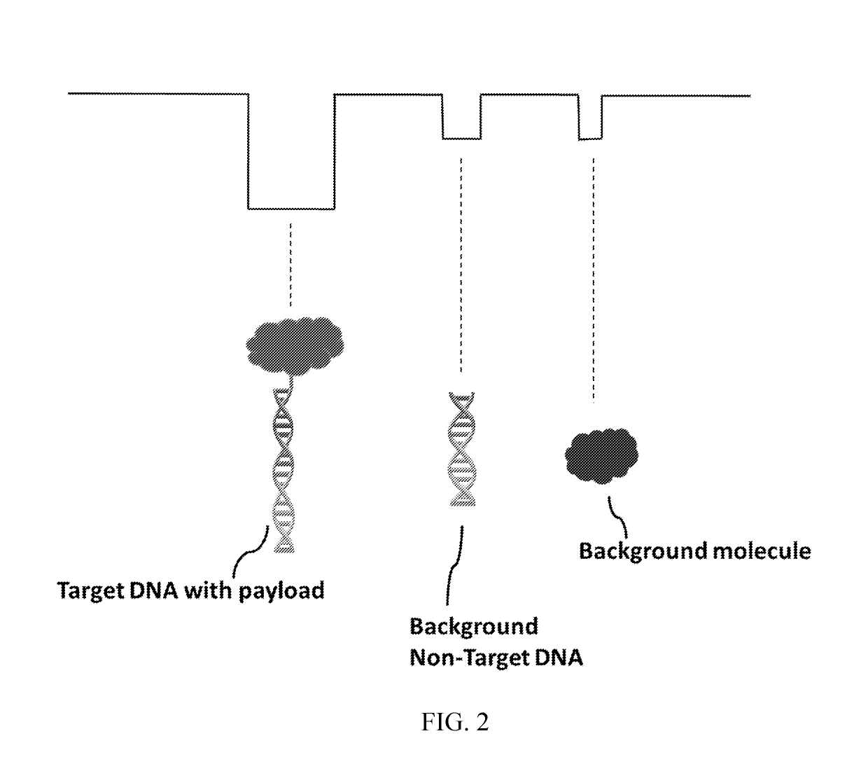

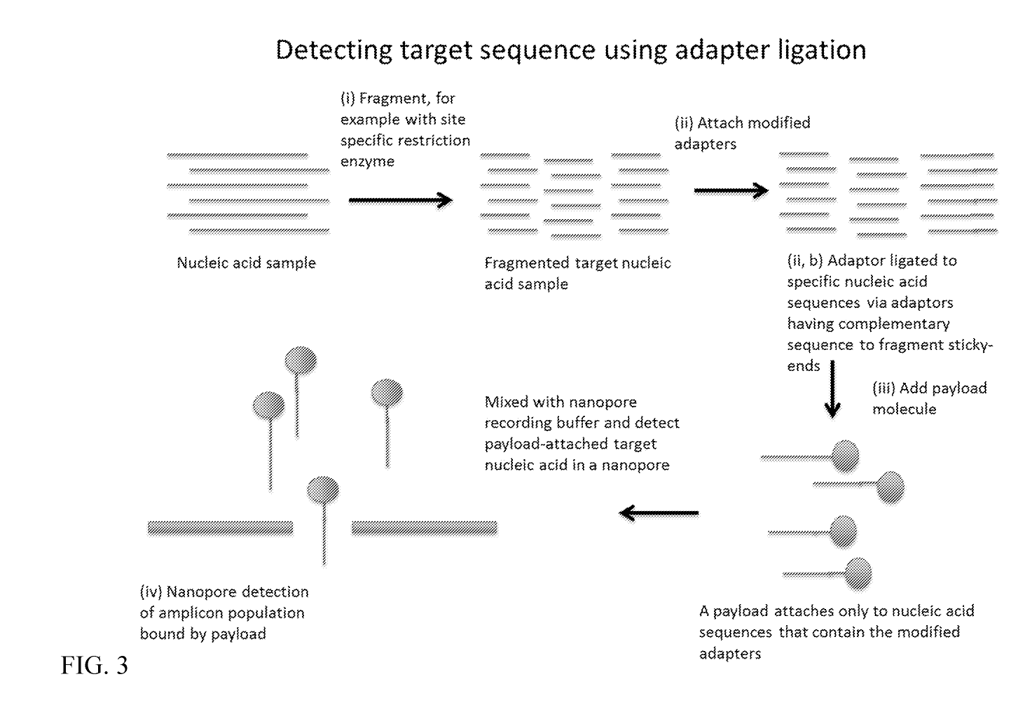

The “binding status” of a target sequence, as used herein, refers to whether the target sequence is bound to a probe. Essentially, the binding status is either bound or unbound. Either, (i) the target sequence is free and not bound to a probe (ii) the target sequence is bound to a probe. FIG. 2 shows representative changes to current through a nanopore due to the presence of target sequence bound to a payload, unbound background non-target DNA, and other background molecules captured in and translocating through the nanopore. Probes of different sizes or having different probe binding sites can be used to give additional current profiles to enable more than one target sequence to be detected in a sample, either on the same polynucleotide or on different polynucleotides.

Detection of the binding status of a target sequence can be carried out by various methods. In one aspect, by virtue of the different sizes of the target sequence at each status (i.e. occupied or unoccupied), when the target sequence passes through the pore, the different sizes result in different currents across the pore. In this respect, no separate sensor is required for the detection, as the electrodes, which are connected to a power source and can detect the current, can serve the sensing function. The two electrodes, therefore, can serve as a “sensor.”

In some aspects, a payload molecule can be added to the probe to facilitate detection. This payload molecule can be already attached to the probe, or can be capable of binding to the probe or polynucleotide/probe complex. In one aspect, the payload molecule includes a charge, either negative or positive, to facilitate detection in a nanopore via an electrical signal, such as current. In another aspect, the payload molecule adds size to facilitate detection via an electrical signal. In another aspect, the payload molecule includes a detectable label, such as a fluorophore.

In some embodiments, the probe comprises a payload binding moiety adapted to bind to said payload molecule. The binding interaction between the payload binding moiety and the payload molecule can be covalent or non-covalent. In some embodiments, the non-covalent binding interaction is characterized as a hydrogen bond, an ionic bond, a van der Waals interaction, a hydrophobic interaction, a cation-pi interaction, a planar stacking interaction, or a metallic bond.

In this context, an identification of a bound status (ii) indicates that the target is bound to a probe. In other words, the target sequence is detected.

In some embodiments, target sequence-specific detection and/or quantification in a nanopore can be performed using the following method (also depicted in FIG. 1):

A sample suspected of containing a target polynucleotide is obtained. The sample is treated to fragment polynucleotides in the sample. This treatment can be exposure to shearing conditions, or exposure to enzymatic cleavage, such as restriction enzymes. The cleavage can be site-specific to facilitate detection of a target sequence. After fragmentation, the sample is contacted with PNA probes (or other suitable probes) capable of binding to a specific target sequence on a fragmented polynucleotide. The PNA probes are bound to a payload binding moiety, or comprise a payload molecule binding moiety which will be bound to the payload binding moiety before detection in a nanopore device. Then, the sample is placed in a nanopore device and a voltage applied to induce translocation of polynucleotide through the nanopore.

The flow of current through the nanopore over time is collected using sensors in the nanopore device. This data is then analyzed to determine the presence or absence of current signatures associated with a polynucleotide target sequence bound to a probe-payload complex, i.e., a polynucleotide-probe complex. Quantification of the amount of target sequence in the sample can also be performed by comparing the capture rate (or other method of event quantification) of the polynucleotide-probe complex in a nanopore with a reference linking the capture rate to the concentration under specified conditions.

Fragmentation

In practical applications of detecting target sequences in a sample obtained from an organism or an environment, the sample can contain DNA exceeding a million base pairs in length, and also contain a significant number of background molecules. Detecting a target sequence among this type of sample poses a significant challenge. For a commercial nanopore technology to be viable and simple, the method for target sequence detection applications must be tolerant to background molecules in a variety of forms. This is particularly true if fragmentation of the sample, and any sequence-specific labeling, occurs directly in the chamber adjacent to the nanopore, just prior to or during nanopore sensing.

Herein we describe, in some embodiments, a method that permits detection and/or quantitation of any target polynucleotide sequence from within the total population of fragmented DNA molecules, without requiring a purification step to remove any background molecules prior to nanopore measurement. Background molecules can include non-target DNA from the fragmentation, and any reagents or molecules utilized with chemistries to add payload molecules to the target sequence-containing DNA fragments, wherein the payload molecule permits selective detection of the target sequence-containing DNA fragments using the nanopore sensor.

In some embodiments, described herein is a method for detecting target polynucleotide sequences with a nanopore by attaching a probe and/or a payload molecule to enable discrimination from background molecules, i.e., all molecules that are not the target nucleic acid. The method does not require nucleic acid purification at any step, which simplifies the device infrastructure required to implement the method. The methods described herein are compatible with a range of nanopore sizes and geometries, and can be implemented in an inexpensive and portable form factor. The method also permits quantitation (i.e., concentration estimation) of the nucleic acid comprising the target sequence in the chamber adjacent to the nanopore sensor.

In some embodiments, the polynucleotide comprising the target sequence is fragmented, either specifically (e.g., via a restriction enzyme) or non-specifically (e.g., via e.g., shearing). This is followed by binding a probe to the fragment comprising the target sequence. This can be done, for example, via ligation of a probe to the end of a fragmented sequence, or through sequence-specific binding to a target sequence of a polynucleotide. In some embodiments, the probe comprises a payload molecule. In some embodiments, the probe comprises a payload binding moiety for binding a payload molecule to the probe, thereby conjugating the payload molecule with the target sequence. In some embodiments, sequence-specific shearing is achieved through the use of restriction enzymes, CRISPR technology, or another shearing method known in the art.

In some embodiments, the polynucleotide fragment comprising the target sequence binds to the probe via a ligation reaction. In some embodiments, the ligation reaction binds a terminal end of a polynucleotide fragment to a probe. In some embodiments, the ligation reaction binds the probe to the fragment, wherein the probe is adapted to specifically bind a payload molecule via a payload molecule binding moiety. In some embodiments, probes can be ssDNA, dsDNA, ssRNA, dsRNA, DNA/RNA hybrids, PNA, or LNA. In some embodiments, the probe and the payload molecule are connected via a covalent bond, or non-covalent bond, e.g. a hydrogen bond, an ionic bond, a van der Waals force, a hydrophobic interaction, a cation-pi interaction, a planar stacking interaction, or a metallic bond.

In certain embodiments, fragmentation and/or binding of the polynucleotide comprising the target sequence to the probe is performed within one or more of the volumes within the said device. In some embodiments, background molecules due to fragmentation and/or binding steps are present in the volume during detection of target sequences using the nanopore device.

An advantage of our fragmentation and probe binding approach is the specificity of the signature generated by the target sequence in a nanopore, allowing discrimination from a large population of background molecules. Thus, in some embodiments, the polynucleotide comprising the target sequence is detected from a crude sample that has not been purified after obtaining the sample from the source (e.g., a source organism or environment).

In some embodiments, two or more payload molecules are attached to each nucleic acid molecule comprising the target sequence. In some embodiments, a plurality of unique target-probe complexes each bound to a different payload molecule can be detected with the nanopore sensor, the different payload molecules adapted to allow discrimination between target sequences in a nanopore for multiplexing.

In certain preferred embodiments, an estimate for the concentration of the polynucleotide comprising the target sequence can be determined from an aggregated set of sensor measurements. In some embodiments, the measurements are compared to a reference to determine a concentration or fractional abundance of the polynucleotide comprising the target sequence.

Probe Specificity

In some aspects, the method further comprises using probes that bind specifically to a sufficiently long target sequence so that they are capable of binding to only one unique sequence in the target population, but also have the ability to not bind to the target site if only a single base pair mismatch is present. This discrimination is possible, for example, when using probes comprising PNA. A 20 bp gamma-PNA probe is able to efficiently bind to a perfectly matched target sequence, but binding is abrogated when the target sequence and probe sequence differ by only one base (Strand-Invasion of Extended, Mixed-Sequence B-DNA by γPNAs, G. He, D. Ly et al., J Am Chem Soc. 2009 Sep. 2; 131(34): 12088-12090. doi:10.1021/ja900228j). When considering the human genome that contains 3.1 billion bases, a 20 base pair sequence is likely to randomly occur 0.003 times. Thus, a 20 base pair probe designed to bind to a specific sequence under investigation is very unlikely to bind to an undesired location and provide a false positive. Therefore, in some embodiments, the target sequence is at least 20 base pairs in length.

Cleavable Payload Molecules

In some aspects, probes comprise payload molecules that allow detection by a sensor, but they are attached to the probe using a cleavable linker. Thus a set of probes that can be distinguished from each other in the nanopore are bound to a target bearing polynucleotide. Once that set of probes is detected in the nanopore, the features are cleaved off and a new set of probes are added that also have cleavable detection feature. The add/cleave/wash cycle can be continued until all sequence information is extracted from a captured target molecule. Example of molecules that aid in probe detection are discussed above. Examples of cleavable linkers are reductant cleavable linkers (disulfide linkers cleaved by TCEP), acid cleavable linker (hydrazone/hydrazide bonds), amino acid sequences that are cleaved by proteases, nucleic acid linkers that are cleaved by endonucleases (sites specific restriction enzymes), base cleavable linkers, or light cleavable linkers [Leriche, Geoffray, Louise Chisholm, and Alain Wagner. “Cleavable linkers in chemical biology.” Bioorganic & medicinal chemistry 20, no. 2 (2012): 571-582.]

Probe Molecules

Probes as used herein are understood to be capable of specifically binding to a site on a polynucleotide, wherein the site is characterized by the sequence or structure. A probe molecule can be detected or quantitated by virtue of its binding to the target sequence-bearing polynucleotide, and capture and translocation of the complex through a nanopore. Examples of probe molecules include a PNA (protein nucleic acid), bis-PNA, gamma-PNA, a PNA-conjugate that increases size or charge of PNA. Other examples of probe molecules are from the group consisting of a natural or recombinant protein, protein fusion, DNA binding domain of a protein, peptide, a nucleic acid, oligo nucleotide, TALEN, CRISPR, a PNA (protein nucleic acid), bis-PNA, gamma-PNA, a PNA-conjugate that increases size, charge, fluorescence, or functionality (e.g. oligo labeled), or any other PNA derivatized polymer, and a chemical compound.

In some aspects, the probe comprises a γ-PNA. γ-PNA has a simple modification in a peptide-like backbone, specifically at the γ-position of the N-(2-aminoethyl)glycine backbone, thus generating a chiral center (Rapireddy S., et al., 2007. J. Am. Chem. Soc., 129:15596-600; He G, et al., 2009, J. Am. Chem. Soc., 131:12088-90; Chema V, et al., 2008, Chembiochem 9:2388-91; Dragulescu-Andrasi, A., et al., 2006, J. Am. Chem. Soc., 128:10258-10267). Unlike bis-PNA, γ-PNA can bind to dsDNA without sequence limitation, leaving one of the two DNA strands accessible for further hybridization.

In some aspects, the function of the probe is to hybridize to a polynucleotide with a target sequence by complementary base pairing to form a stable complex. The PNA molecule may additionally be bound to additional molecules to form a complex has sufficiently large cross-section surface area to produce a detectable change or contrast in signal amplitude over that of the background, which is the mean or average signal amplitude corresponding to sections of non-probe-bound polynucleotide.

The stability of the binding of the polynucleotide target sequence to the PNA molecule is important in order for it to be detected by a nanopore device. The binding stability must be maintained throughout the period that the target-bearing polynucleotide is being translocated through the nanopore. If the stability is weak, or unstable, the probe can separate from the target polynucleotide and will not be detected as the target-bearing polynucleotide threads through the nanopores.

In a particular embodiment, an example of a probe is a PNA bound to a payload molecule or to a molecule comprising a binding moiety adapted to bind to a payload molecule in which the PNA specifically recognizes a nucleotide sequence and the payload molecule increases the sensitivity of detection in a nanopore device. Different payload molecules with size/shape/charge differences may also be used to discriminate between different PNA-payload complexes bound to their respective target sequences in a nanopore.

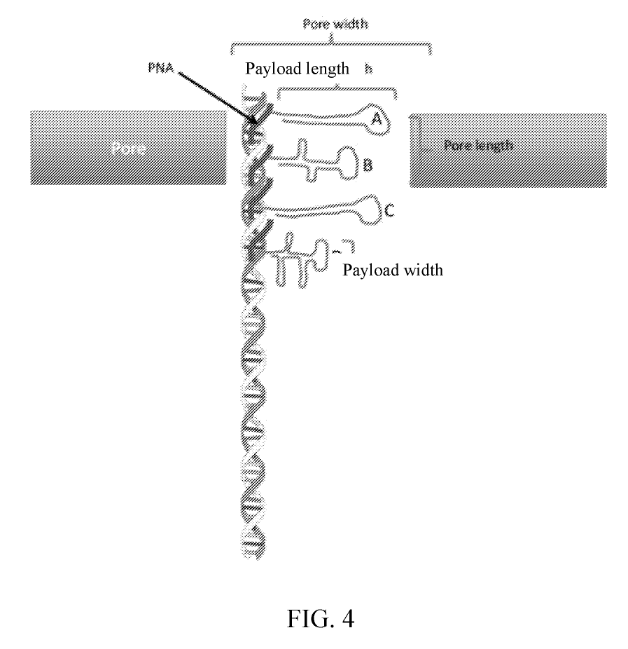

As illustrated in FIG. 4, probes A, B, C and D each specifically binds to a site on a DNA molecule, and these probes can be identified and distinguished from each other by the width, length, size and/or charge of the bound payload molecule. If their corresponding sites are denoted as A, B, C and D, respectively, then identification of the ligands leads to revelation of those DNA sequences, A-B-C-D. Note that the probes can each be bound to unique polynucleotides, as is more likely with fragmented polynucleotides.

Different reactive payload binding moieties may be incorporated into the probes to provide a chemical handle to which payload molecules may bind. Examples of reactive payload binding moieties include, but are not limited to, primary amines, carboxylic acids, ketones, amides, aldehydes, boronic acids, hydrazones, thiols, maleimides, alcohols, and hydroxyl groups, and biotin.

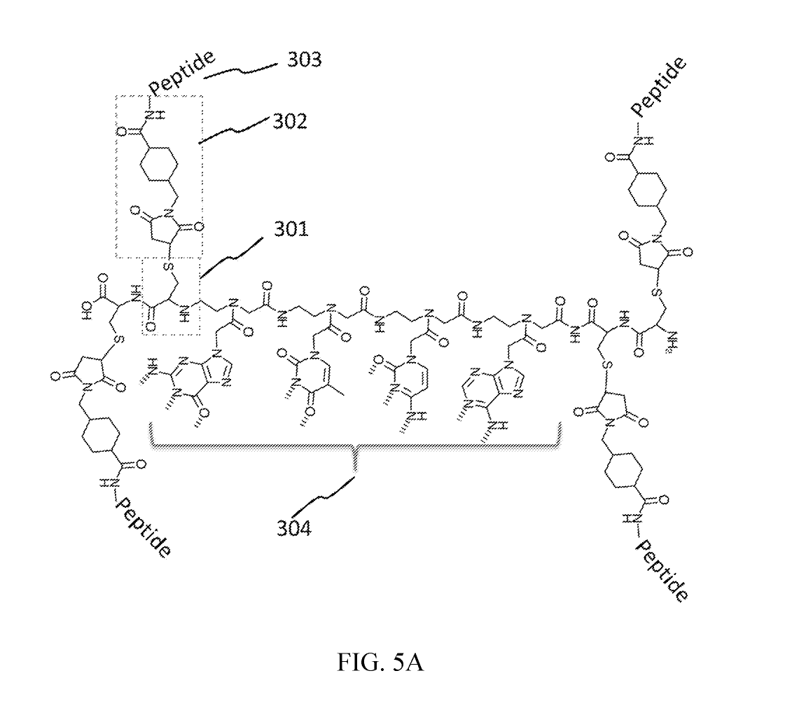

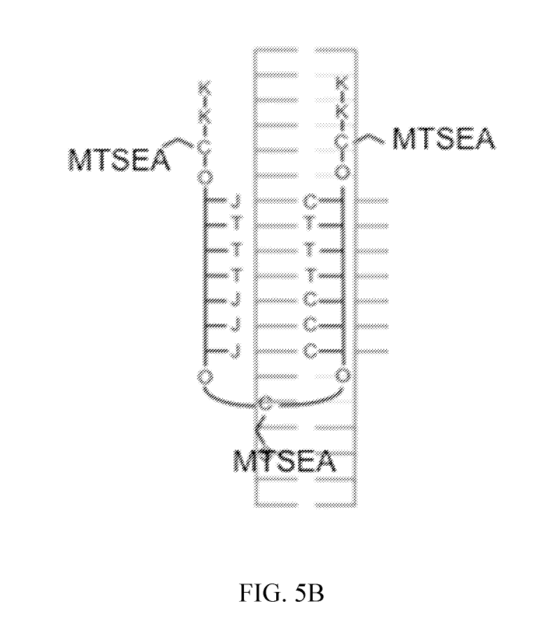

FIG. 5A shows a PNA probe that has been modified as to increase its charge, and therefore facilitate detection by a nanopore. Specifically, this probe, which binds to the target DNA sequence by complementary base pairing and Hoogsteen base pairing between the bases on the PNA molecule and the bases in the target DNA sequence (i.e., the target sequence), has cysteine residues incorporated into the backbone, which provide a free thiol payload binding moiety to attach one or more payload molecules. Here, the cysteine is bound to a peptide 2-aminoethylmethanethiosulfonate (MTSEA) through a maleimide linker, which provides a means to enhance detection in a nanopore device of whether the probe is bound to its target sequence since the payload molecule increases the probe's charge. This greater charge results in a greater change in current flow through the pore during translocation as compared to a PNA probe without the payload molecule bound.

In some aspects, to increase the specificity and sensitivity of discrimination between the polynucleotide-probe complex and other background molecules present in the sample, modification can be made to the pseudo-peptide backbone to change the overall size of the PNA probe. See, e.g., FIG. 5B, which shows a PNA that has cysteine residues (301) incorporated that are modified with a succinimidyl 4-(N-maleimidomethyl)cyclohexane-1-carboxylate (SMCC) linker (302) to enable conjugation to peptides (303) through the N-terminal amine of the peptide. In addition to adding charge directly to the probe (e.g., as in FIG. 5A), selection of more charged amino acids instead of non-polar amino acids can serve to increase the charge of PNA. Payload molecules, such as small particle, molecules, protein, peptides, or polymers (e.g. PEG) can be bound to the pseudo-peptide backbone to enhance the bulk or cross-sectional surface area of the polynucleotide-probe complex. Enhanced bulk serves to improve the signal amplitude contrast so that any differential signal resulting from the increased bulk can be easily detected, even in the presence of a significant amount of background molecules, e.g., as in a non-purified sample. Examples of small particle, molecules, protein, or peptides that can act as payload molecules to bind to the pseudo-peptide backbone include but are not limited to alpha-helical forming peptides, nanometer-sized gold particles or rods (e.g. 3 nm), quantum dots, polyethylene glycol (PEG). Methods of conjugation (i.e., binding) of molecules are well known in the art, e.g. in U.S. Pat. Nos. 5,180,816, 6,423,685, 6,706,252, 6,884,780, and 7,022,673, which are hereby incorporated by reference in their entirety.

The embodiments above describe binding of payload molecules to a PEG probe through cysteine residues, however other residues can also be used. For example, Lysine residues are easily interchanged with cysteine residues to enable linkage chemistry using NETS-esters and free amines. Also, PEG can easily be interchanged with other bulk-adding constituents, such as dendrons, beads, or rods. Between the bifunctional linker and the PNA, or to directly couple the Dendron. Someone skilled in the art would recognize the flexibility of this system in that the amino acid can be changed and linkage chemistry modified for that particular amino acid, e.g. serine reactive isocyanates. Some examples of linkage chemistry that can be used for this reaction is listed in the table below.

TABLE 1
Linkage Chemistry

| Reactive Group | Target Functional Group |
|---|---|
| aryl azide | nonselective or primary amine |
| carbodiimide | amine/carboxyl |
| hydrazide | carbohydrate |
| hydroxymethyl phosphine | amine |
| imidoester | amine |
| isocyanate | hydroxyl |
| carbonyl | hydrazine |
| maleimide | sulfhydryl |
| NHS-ester | amine |
| PFP-ester | amine |
| psoralen | thymine |
| pyridyl disulfide | sulfhydryl |
| vinyl sulfone | sulfhydryl amine, hydroxyl |

FIGS. 1, 2, 5A, 5B and 6A show PNA probes that have been modified to increase probe size, or to bind to a payload molecule, an ssDNA oligomer, a fluorophores, or a charge. In some embodiments, the payload molecules increase size to facilitate detection or to discriminate from other probes during a multiplex target sequence detection.

In some embodiments, the binding moiety comprises a chemical handle to bind the probe to the payload molecule. A common method for incorporating the chemical handles are to include a specific amino acid into the backbone of the probe. Examples include, but are not limited to, cysteines (provide thiolates), lysines (provides free amines), threonine (provides hydroxyl), glutamate and aspartate (provides carboxylic acids).

Different types of payload molecules can be added using the binding moieties. These include payload molecules that:
1. increase the size of the probe, e.g. biotin/streptavidin, peptide, nucleic acid;
2. change the charge of the probe, e.g. a charged peptide (6×HIS), or protein (e.g., charybdotoxin), or small molecule or peptide (e.g. MTSET);
3. change or add fluorescence to the probe, e.g. common fluorophores, FITC, Rhodamine, Cy3, Cy5; or
4. provide an epitope or interaction site for binding a third molecule, e.g. peptides for binding antibody.

Quantification

Enhanced detection of the target fragment via the payload probe detection mechanism, which allows us to distinguish the event signature of the target-containing fragments from all other detected events provides a value for relative abundance of the target that is not reflected purely by the capture and detection rate of all molecules in the nanopore. Thus, in general, it may not be possible to identify the correct target:non-target ratio from the detected event ratio of the payload-bound targets vs. non-targets, since the ratio of detected event types is significantly different from the ratio of molecule types in the chamber of molecules.

Thus, in order to determine the concentration of a DNA/PNA-payload population, and therefore the target-containing fragment, we provide herein methods to compensate for the enhanced detection of the target molecule. In one embodiment, provided herein is a method to quantify the target molecule in a sample using a secondary molecule type that is detectable in the nanopore with a unique event signature (either alone or bound to a probe/probe-payload complex) from the target molecule-payload complex and from non-target fragments.

We have previously derived a method for quantifying the fractional amount of a target molecule (e.g., a payload-bound target fragment) relative to a secondary molecule (U.S. Provisional Application No. 62/412,221, filed Oct. 24, 2016 incorporated in its entirety by reference.

In some embodiments, a secondary molecule type that has a unique event profile distinguished from non-target fragments and from payload-bound targets is introduced at known concentrations. The secondary molecule at known concentration can be mixed with the prepared sample (containing non-target fragments and the payload-bound target fragments). The mixture can then be measured on the nanopore. Prior to or after measuring the mixture on the nanopore, a control mixture that contains a known concentration of payload-bound target molecules and a known concentration of secondary molecules can also be measured on the nanopore. The control mixture can use equal concentrations of payload-bound target fragments and secondary molecules (i.e., a 1:1 ratio) or any other ratio.

In some embodiments, the ratio of the secondary molecule to the target molecule in the control concentration is near the anticipated ratio of secondary molecule to target in the unknown sample, although this may not be known ahead of time. If a likely range of the unknown is identified, the control or secondary molecule concentrations can be chosen within the expected range. Separate from the control mixture, isolated controls may be run, including secondary molecules alone, and the target-payload molecule alone. Such isolated controls can be used instead of the control mixture, or in addition to the control mixture, and collectively the controls (isolated and mixtures) can improve the determination of fractional abundance or target concentration in a sample.

Multiplexing

In some embodiments, rather than including probes of the same kind, a collection of different probes can be added that each bind to a unique target sequence.

Within such a setting, multiple different probes can be used to detect multiple different target sequences within the same or different target bearing polynucleotides. By using probes that each provide a unique current profile (e.g., by differing in size), the present technology can detect different target sequences within the same molecule, providing a means for multiplexing target sequence detection. Further, by enumerating how many of each unique probes are bound, number of each target (or copy number) can be determined. By tuning conditions that impact the bindings, the system can obtain more detailed binding dynamic information.

Similarly, multiplexing can be accomplished by having a collection of probes with differing attributes and mixed-and-matched in any number of combination, the only requirement is that probes that bind to a different sequence are discernable from each other.

Nanopore Devices

A nanopore device, as provided, includes a pore that forms an opening in a structure separating an interior space of the device into two volumes, and is configured to identify objects (for example, by detecting changes in parameters indicative of objects) passing through the pore, e.g., with a sensor. Nanopore devices used for the methods described herein are also disclosed in PCT Publication WO/2013/012881, incorporated by reference in entirety.

The pore(s) in the nanopore device are of a nano scale or micro scale. In one aspect, each pore has a size that allows a small or large molecule or microorganism to pass. In one aspect, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, 90 nm, or 100 nm in diameter.

In one aspect, the pore is no more than about 100 nm in diameter. Alternatively, the pore is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm in diameter.

In some aspects, each pore is at least about 100 nm, 200 nm, 500 nm, 1000 nm, 2000 nm, 3000 nm, 5000 nm, 10000 nm, 20000 nm, or 30000 nm in diameter. In one aspect, the pore is no more than about 100000 nm in diameter. Alternatively, the pore is no more than about 50000 nm, 40000 nm, 30000 nm, 20000 nm, 10000 nm, 9000 nm, 8000 nm, 7000 nm, 6000 nm, 5000 nm, 4000 nm, 3000 nm, 2000 nm, or 1000 nm in diameter.

In one aspect, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some aspects, the pore(s) in the nanopore device are of a larger scale for detecting large microorganisms or cells. In one aspect, each pore has a size that allows a large cell or microorganism to pass. In one aspect, each pore is at least about 100 nm in diameter. Alternatively, each pore is at least about 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, 1600 nm, 1700 nm, 1800 nm, 1900 nm, 2000 nm, 2500 nm, 3000 nm, 3500 nm, 4000 nm, 4500 nm, or 5000 nm in diameter.

In one aspect, the pore is no more than about 100,000 nm in diameter. Alternatively, the pore is no more than about 90,000 nm, 80,000 nm, 70,000 nm, 60,000 nm, 50,000 nm, 40,000 nm, 30,000 nm, 20,000 nm, 10,000 nm, 9000 nm, 8000 nm, 7000 nm, 6000 nm, 5000 nm, 4000 nm, 3000 nm, 2000 nm, or 1000 nm in diameter.

In one aspect, the pore has a diameter that is between about 100 nm and about 10000 nm, or alternatively between about 200 nm and about 9000 nm, or between about 300 nm and about 8000 nm, or between about 400 nm and about 7000 nm, or between about 500 nm and about 6000 nm, or between about 1000 nm and about 5000 nm, or between about 1500 nm and about 3000 nm.

In some aspects, the nanopore device further includes means to move a polynucleotide across the pore and/or means to identify objects that pass through the pore. Further details are provided below, described in the context of a two-pore device.

Compared to a single-pore nanopore device, a two-pore device can be more easily configured to provide good control of speed and direction of the movement of the polynucleotide across the pores.

In certain embodiments, the nanopore device includes a plurality of chambers, each chamber in communication with an adjacent chamber through at least one pore. Among these pores, two pores, namely a first pore and a second pore, are placed so as to allow at least a portion of a polynucleotide to move out of the first pore and into the second pore. Further, the device includes a sensor capable of identifying the polynucleotide during the movement. In one aspect, the identification entails identifying individual components of the polynucleotide. In another aspect, the identification entails identifying fusion molecules and/or target analytes bound to the polynucleotide. When a single sensor is employed, the single sensor may include two electrodes placed at both ends of a pore to measure an ionic current across the pore. In another embodiment, the single sensor comprises a component other than electrodes.

In one aspect, the device includes three chambers connected through two pores. Devices with more than three chambers can be readily designed to include one or more additional chambers on either side of a three-chamber device, or between any two of the three chambers. Likewise, more than two pores can be included in the device to connect the chambers.

In one aspect, there can be two or more pores between two adjacent chambers, to allow multiple polynucleotides to move from one chamber to the next simultaneously. Such a multi-pore design can enhance throughput of polynucleotide analysis in the device.

In some aspects, the device further includes means to move a polynucleotide from one chamber to another. In one aspect, the movement results in loading the polynucleotide across both the first pore and the second pore at the same time. In another aspect, the means further enables the movement of the polynucleotide, through both pores, in the same direction.

For instance, in a three-chamber two-pore device (a “two-pore” device), each of the chambers can contain an electrode for connecting to a power supply so that a separate voltage can be applied across each of the pores between the chambers.

In accordance with an embodiment of the present disclosure, provided is a device comprising an upper chamber, a middle chamber and a lower chamber, wherein the upper chamber is in communication with the middle chamber through a first pore, and the middle chamber is in communication with the lower chamber through a second pore. Such a device may have any of the dimensions or other characteristics previously disclosed in U.S. Publ. No. 2013-0233709, entitled Dual-Pore Device, which is herein incorporated by reference in its entirety.

In some embodiments, the device includes an upper chamber, a middle chamber, and a lower chamber. The chambers are separated by two separating layers or membranes each having a separate pore. Further, each chamber contains an electrode for connecting to a power supply. The annotation of upper, middle and lower chamber is in relative terms and does not indicate that, for instance, the upper chamber is placed above the middle or lower chamber relative to the ground, or vice versa.

The two pores can be arranged in any position so long as they allow fluid communication between the chambers and have the prescribed size and distance between them. In one aspect, the pores are placed so that there is no direct blockage between them.

In one aspect, the device is connected to one or more power supplies. In some aspects, the power supply includes a voltage-clamp or a patch-clamp, which can supply a voltage across each pore and measure the current through each pore independently. In this respect, the power supply and the electrode configuration can set the middle chamber to a common ground for both power supplies. In one aspect, the power supply or supplies are configured to apply a first voltage V₁ between the upper chamber and the middle chamber, and a second voltage V₂ between the middle chamber and the lower chamber.

In some aspects, the first voltage V₁ and the second voltage V₂ are independently adjustable. In one aspect, the middle chamber is adjusted to be a ground relative to the two voltages. In one aspect, the middle chamber comprises a medium for providing conductance between each of the pores and the electrode in the middle chamber. In one aspect, the middle chamber includes a medium for providing a resistance between each of the pores and the electrode in the middle chamber. Keeping such a resistance sufficiently small relative to the nanopore resistances is useful for decoupling the two voltages and currents across the pores, which is helpful for the independent adjustment of the voltages.

Adjustment of the voltages can be used to control the movement of charged particles in the chambers. For instance, when both voltages are set in the same polarity, a properly charged particle can be moved from the upper chamber to the middle chamber and to the lower chamber, or the other way around, sequentially. In some aspects, when the two voltages are set to opposite polarity, a charged particle can be moved from either the upper or the lower chamber to the middle chamber and kept there.

The adjustment of the voltages in the device can be particularly useful for controlling the movement of a large molecule, such as a charged polynucleotide, that is long enough to cross both pores at the same time. In such an aspect, the direction and the speed of the movement of the molecule can be controlled by the relative magnitude and polarity of the voltages as described below.

The device can contain materials suitable for holding liquid samples, in particular, biological samples, and/or materials suitable for nanofabrication. In one aspect, such materials include dielectric materials such as, but not limited to, silicon, silicon nitride, silicon dioxide, graphene, carbon nanotubes, TiO₂, HfO₂, Al₂O₃, or other metallic layers, or any combination of these materials. In some aspects, for example, a single sheet of graphene membrane of about 0.3 nm thick can be used as the pore-bearing membrane.

Devices that are microfluidic and that house two-pore microfluidic chip implementations can be made by a variety of means and methods. For a microfluidic chip comprised of two parallel membranes, both membranes can be simultaneously drilled by a single beam to form two concentric pores, though using different beams on each side of the membranes is also possible in concert with any suitable alignment technique.

In one aspect, the device includes a microfluidic chip (labeled as “Dual-core chip”) is comprised of two parallel membranes connected by spacers. Each membrane contains a pore drilled by a single beam through the center of the membrane. Further, the device preferably has a Teflon® housing for the chip. The housing ensures sealed separation of Chambers A-C and provides minimal access resistance for the electrode to ensure that each voltage is applied principally across each pore.

By virtue of the voltages present at the pores of the device, charged molecules can be moved through the pores between chambers. Speed and direction of the movement can be controlled by the magnitude and polarity of the voltages. Further, because each of the two voltages can be independently adjusted, the direction and speed of the movement of a charged molecule can be finely controlled in each chamber.

One example concerns a charged polynucleotide, such as a DNA, having a length that is longer than the combined distance that includes the depth of both pores plus the distance between the two pores. For example, a 1000 by dsDNA is about 340 nm in length, and would be substantially longer than the 40 nm spanned by two 10 nm-deep pores separated by 20 nm. In a first step, the polynucleotide is loaded into either the upper or the lower chamber. By virtue of its negative charge under a physiological condition at a pH of about 7.4, the polynucleotide can be moved across a pore on which a voltage is applied. Therefore, in a second step, two voltages, in the same polarity and at the same or similar magnitudes, are applied to the pores to move the polynucleotide across both pores sequentially.

At about the time when the polynucleotide reaches the second pore, one or both of the voltages can be changed. Since the distance between the two pores is selected to be shorter than the length of the polynucleotide, when the polynucleotide reaches the second pore, it is also in the first pore. A prompt change of polarity of the voltage at the first pore, therefore, will generate a force that pulls the polynucleotide away from the second pore.

Assuming that the two pores have identical voltage-force influence and |V₁|=|V₂|+δV, the value δV>0 (or <0) can be adjusted for tunable motion in the V₁| (or V₂) direction. In practice, although the voltage-induced force at each pore will not be identical with V₁=V₂, calibration experiments can identify the appropriate bias voltage that will result in equal pulling forces for a given two-pore chip; and variations around that bias voltage can then be used for directional control.

If, at this point, the magnitude of the voltage-induced force at the first pore is less than that of the voltage-induced force at the second pore, then the polynucleotide will continue crossing both pores towards the second pore, but at a lower speed. In this respect, it is readily appreciated that the speed and direction of the movement of the polynucleotide can be controlled by the polarities and magnitudes of both voltages. As will be further described below, such a fine control of movement has broad applications.

Sensors

In certain embodiments, the nanopore devices of the present invention include one or more sensors to carry out the identification of a target sequence in the nanopore using the methods described herein.

The sensors used in the device can be any sensor suitable for identifying the target sequence of polynucleotide via translocation of a polynucleotide-probe complex through the nanopore. For instance, a sensor can be configured to identify the polynucleotide-probe complex by measuring a current, a voltage, pH, an optical feature or residence time associated with the polynucleotide-probe complex or one or more individual components of the charged polymer. In some embodiments, the sensor includes a pair of electrodes placed at opposing sides of a pore to measure an ionic current through the pore when a molecule or particle, in particular a polynucleotide-probe complex, moves through the nanopore.

In certain embodiments, the sensor measures an optical feature of the polynucleotide-probe complex or a component (or unit) of the polymer. One example of such measurement includes identification by infrared (or ultraviolet) spectroscopy of an absorption band unique to a particular unit.

When residence time measurements are used, they will correlate the size of the unit to the specific unit based on the length of time it takes to pass through the sensing device.

In some embodiments, the sensor is functionalized with reagents that form distinct non-covalent bonds with each of the probes. In this respect, the gap can be larger and still allow effective measuring. For instance, a 5 nm gap can be used to detect a probe/target complex measuring roughly 5 nm. Tunnel sensing with a functionalized sensor is termed “recognition tunneling.” Using a Scanning Tunneling Microscope (STM) with recognition tunneling, a probe bound to a target sequence is easily identified.

Therefore, the methods of the present technology can provide polynucleotide-probe complex delivery rate control for one or more recognition tunneling sites, each positioned in one or both of the nanopore channels or between the pores, and voltage control can ensure that each probe/target complex resides in each site for a sufficient duration for robust identification.

Sensors in the devices and methods of the present disclosure can comprise gold, platinum, graphene, or carbon, or other suitable materials. In a particular aspect, the sensor includes parts made of graphene. Graphene can act as a conductor and an insulator, thus tunneling currents through the graphene and across the nanopore can accurately detect the identity of the polynucleotide-probe complex

In some embodiments, the tunnel gap has a width that is from about 1 nm to about 20 nm. In one aspect, the width of the gap is at least about 1 nm, or alternatively at least about 1.5, 2, 2.5, 3, 3.5, 4, 4.5, 5, 6, 7, 8, 9, 10, 12 or 15 nm. In another aspect, the width of the gap is not greater than about 20 nm, or alternatively not greater than about 19, 18, 17, 16, 15, 14, 13, 12, 11, 10, 9, 8, 7, 6, 5, 4, 3, or 2 nm. In some aspects, the width is between about 1 nm and about 15 nm, between about 1 nm and about 10 nm, between about 2 nm and about 10 nm, between about 2.5 nm and about 10 nm, or between about 2.5 nm and about 5 nm.

In some embodiments, the sensor detects an electrical signal. In some embodiments, the sensor detects a fluorescent signal emitted by said probe and/or payload molecule. A radiation source at the outlet can be used to detect polynucleotide-probe complex-specific signal.

It is to be understood that while the invention has been described in conjunction with the above embodiments, that the foregoing description and following examples are intended to illustrate and not limit the scope of the invention. Other aspects, advantages and modifications within the scope of the invention will be apparent to those skilled in the art to which the invention pertains.

### EXAMPLES

### Example 1—Sequence Specific Probe Synthesis and Target Sequence Binding

In this example, we show the generation of a PNA probe for binding to a target sequence of interest, with features added to the PNA probe to allow for increased sensitivity of detection in a nanopore.

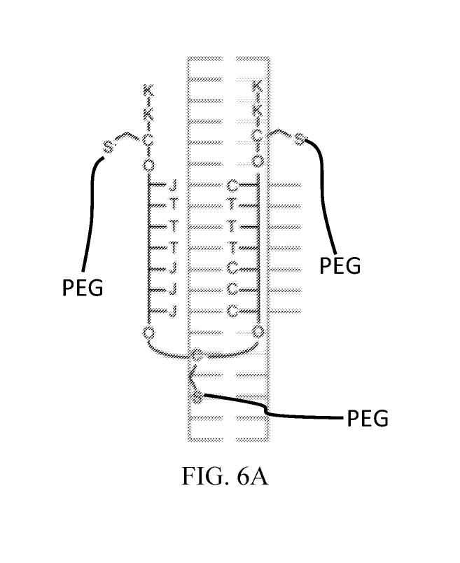

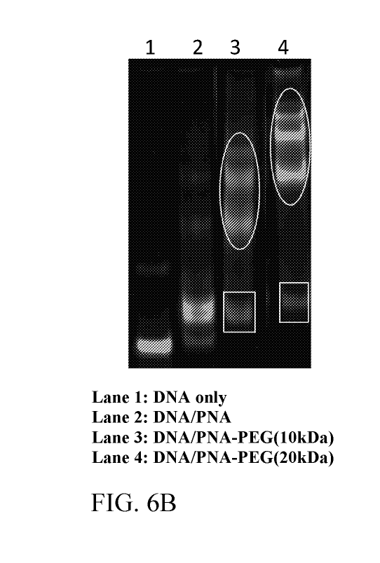

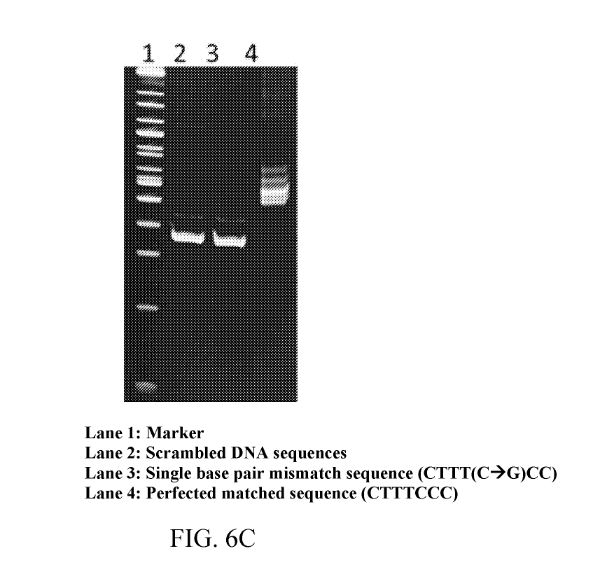

We generated a bisPNA probe containing 3 cysteine residues. The bisPNA probe comprises a sequence of PNA capable of binding to its a DNA sequence comprising a target sequence of CTTTCCC at the location of this target sequence on a target DNA molecule. The bisPNA probe was also labeled with maleimido-PEG-Me at 3 cysteine residues on the bisPNA probe to enhance detection of the probe attached to a target DNA molecule in a nanopore. The PNA-PEG probe was generated by incubating a 100 fold excess of linker (Methyl-PEG(10 kDa)-Maleimide) with bisPNA (Lys-Lys-Cys-PEG3-JTTTJJJ-PEG-Cys-PEG-CCCTTTC-PEG-Cys-Lys-Lys) under reducing conditions. The maleimide portion of the linker reacts with the free thiols in the PNA at pH 7.4, thus creating the PEGylated-PNA. The addition of lysines increases the reagent affinity for its specific cognate DNA sequence thereby allowing it to remain bound under high salt conditions (1 M LiCl). The resulting PNA-PEG probe bound to its target sequence on a dsDNA molecule is shown in FIG. 6A.

To confirm the binding of the DNA-PEG probe to its target sequence on a DNA molecule, we incubated different versions of the PEG probe with DNA and performed a gel shift assay using the resulting solution. For this assay, we ran 4 samples, as shown in FIG. 6B. Lane 1 is DNA only, lane 2 is DNA+PNA, lane 3 is DNA+PNA-PEG (10 kDa), and lane 4 is DNA+PNA-PEG (20 kDa). The upward shift in lanes 2-4 is consistent with the bisPNA species being bound to DNA. The circled species are DNA/PNA-PEG, the boxed species are DNA/PNA in lanes 3 and 4 present as residual PNA (sans PEG) in the labeling experiment. The results of the gel shift assay show complex formation of a DNA containing the target sequence and the PNA probe with a cognate DNA sequence complementary to the target sequence regardless of the attachment of a PEG to the PNA. Thus, we here show successful complex formation of a sequence-specific probe capable of being detected in a nanopore.

We then performed an assay to show specificity of the PNA probe for its target DNA sequence. Here, we incubated a PNA probe (without PEG), with a sample comprising DNA without the target sequence (lane 2), DNA with the target sequence comprising a single base mismatch with the PNA probe (lane 3), and DNA with the complete target sequence (lane 4), and analyzed each sample using a gel shift assay, the results of which are shown in FIG. 6. Lane 1 is a DNA marker. As shown by our results, DNA with an exact target sequence match (lane 4) binds the PNA, while DNA with the target sequence comprising a single base mismatch sequence (lane 3), and DNA without the target sequence (random sequence in place of the target sequence) (lane 2) show no PNA binding. Therefore, the gel shift assay shows that PNA specifically binds to DNA comprising its target sequence, without binding to DNA having even a single mismatch in the target sequence.

### Example 2—Target Sequence Detection in a Nanopore Using a Modified Sequence-Specific Probe

In this example, we show detection of a DNA molecule comprising the target sequence bound to our PEG modified sequence-specific PNA probe.

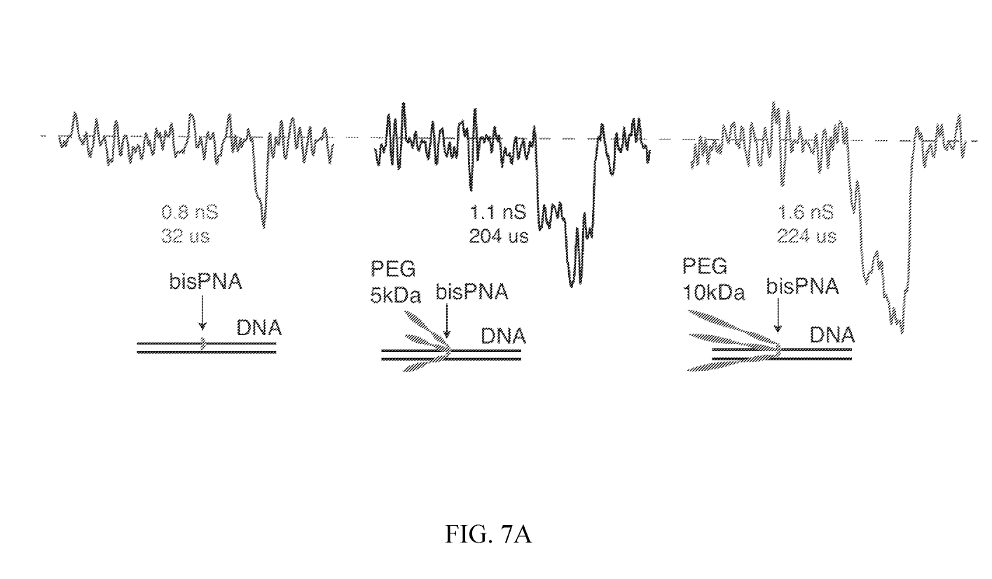

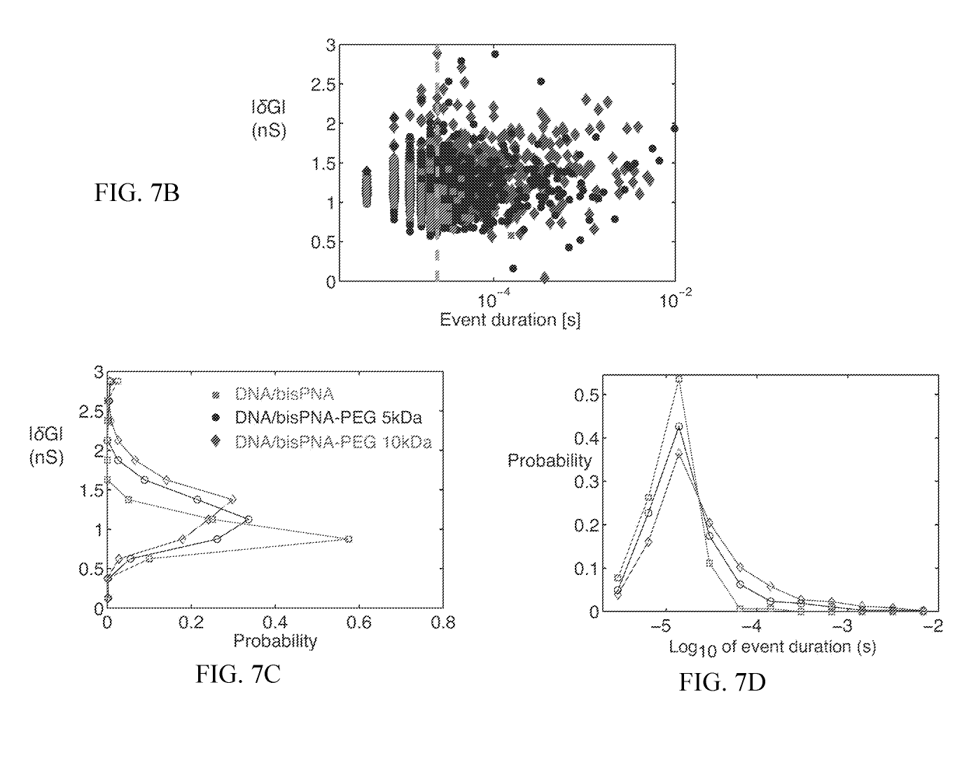

Here we provided three different PNA probes to have different bulkiness based on PEG attachment and PEG length. Three types of probes were used: 1) PNA with no PEG, 2) PNA bound to a 5 kDa PEG, and 3) PNA bound to a 10 kDa PEG. Each probe was mixed with DNA comprising the target sequence and run in the nanopore to observe detection of the DNA bound to the PNA probe. The concentration of each complex in the sample was 2 nM in 1M LiCl buffer. The sample was run in the nanopore device under an applied voltage of 100 mV. The results are shown in FIGS. 7A-7D.

Representative individual events observed are shown for DNA bound to each type of probe in FIG. 7A. The event signature from a DNA/bisPNA event is shown on the left. The event signature from a DNA/bisPNA-PEG complex with up to 3 PEGs bound to each PNA, and PEG sized 5 kDa is shown in the middle. The event signature from a DNA/bisPNA-PEG complex with up to 3 PEGs bound to each PNA, and PEG sized 10 kDa is shown on the right. The event signature for each was measured by current blockade through the nanopore during translocation of the identified complex. In FIG. 7A, molecule depictions show linear PEG and DNA sized to scale for visual comparison. As the probe size (bulk) is increased, the event signature changes.

We analyzed the population of events and generated a scatter plot of mean conductance shift (dG) vs. duration for all events in each data set. We generated a scatter plot of event mean conductance (mean current shift divided by voltage) versus event duration (width) from our experiment as shown in FIG. 7B. The plot shows that DNA/PNA, DNA/PNA-PEG (5 kDa), and DNA/PNA-PEG (10 kDa) give overlapping populations that are distinct based on their event duration and mean conductance. We generated a histogram to show the observed difference in mean conductance shift for each event (dG) between the different complexes (FIG. 7C). We also generated a histogram to show the observed difference in event duration between the different complexes (FIG. 7D).

### Example 3—Detection of a Mutated Cftr Gene Target Sequence in a Nanopore to Detect Human Cystic Fibrosis

We have shown the specificity of binding of our modified PNA probe to a target sequence and the ability to detect the target sequence using the probe in a nanopore device. Here, we look to the use of the modified PNA probe in a nanopore device to detect a disease causing mutation in a sample from a patient, specifically cystic fibrosis.

We generated (according to the methods described in Example 4) a modified PNA probe (PNA-PEG probe) which comprises a PNA molecule that binds specifically to a target DNA sequence comprising a cftr gene with a mutation therein (ΔF508) which causes cystic fibrosis. The PEG bound to the PNA probe was 5 kDa. DNA containing a Cystic Fibrosis disease mutation was incubated with a PEGylated PNA specific for the mutation. The samples were then placed in a nanopore device having a 26 nm pore and translocation events through the nanopore were recorded and analyzed.

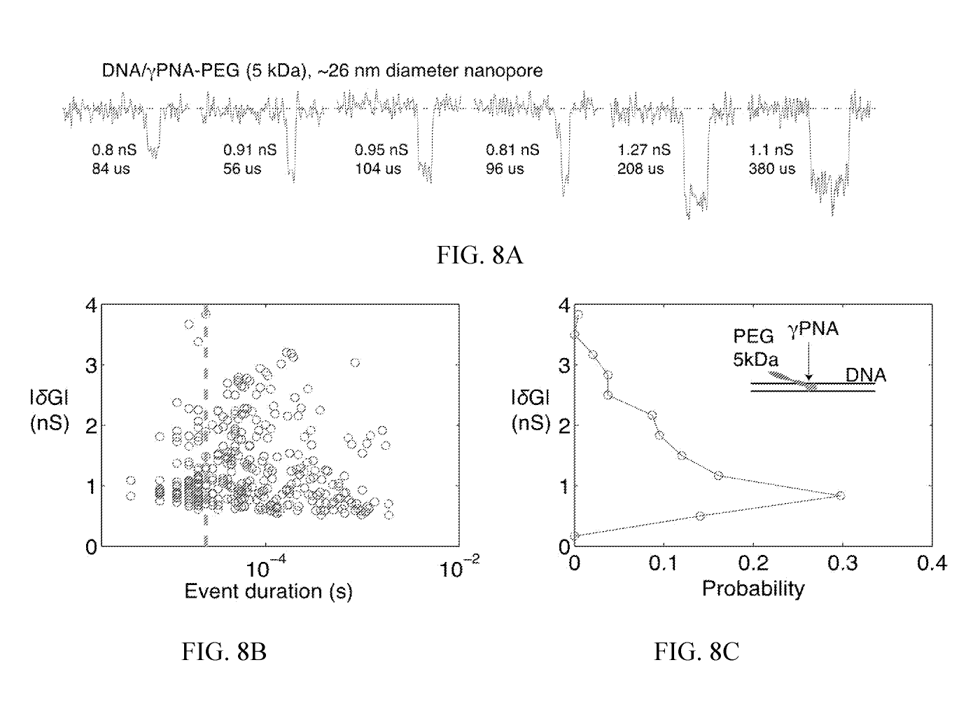

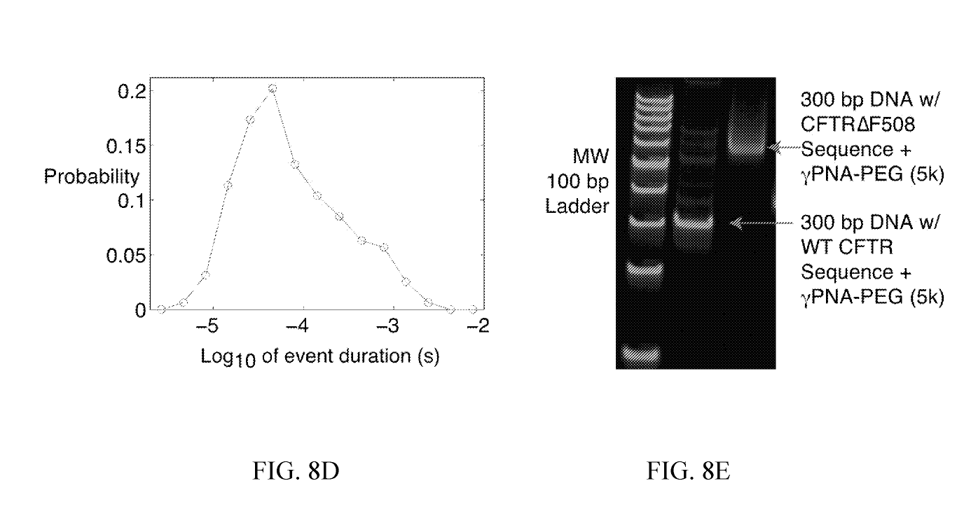

Translocation event signatures correlated with the translocation of a PNA-PEG probe bound to a DNA molecule were observed in the sample with DNA containing the cystic fibrosis causing mutation (ΔF508). Representative event signatures are shown in FIG. 8A. Experiments using sample with DNA only or DNA/PNA only (i.e., no PEG-PNA) gave no definitive translocation events above background, showing the ability of the pore to accurately identify PNA-PEG probe bound to DNA, and the enhancement of detection provided by the modified probes provided herein. For the set of recorded events from a sample with the target mutated gene and the PNA-PEG probe, the events were characterized by mean conductance shift and duration and analyzed. FIG. 8B shows the mean conductance shift v. duration plot for each recorded event. FIG. 8C and FIG. 8D show corresponding histograms to characterize the events detected by mean conductance shift and duration of each event respectively. The analyzed data matched the expected data for a DNA/PNA-PEG (5 kDa) complex translocation through the nanopore, indicating successful binding and identification of the cftr mutation target sequence in the nanopore device.

We also ran a gel shift assay on samples comprising our PNA-PEG(5 kDa) probe specific for the ΔF508 cftr gene mutation with a sample comprising 300 bp DNA with the wild-type cftr sequence (lane 2) and with a sample comprising 300 bp DNA with the ΔF508 cftr gene mutation (lane 3) (FIG. 8E). This data shows that our PNA-PEG probe binds specifically to only the ΔF508 target sequence, but does not bind to the wild-type sequence.

Therefore, we have successfully detected DNA comprising the single base cftr gene mutation (ΔF508), and have here demonstrated the use of our system to detect specific sequences of a polynucleic acid in a sample, including for diagnostic or treatment indications in a human patient.

### Example 4—Infectious Bacteria Detection with the PNA-PEG Probe in a Nanopore

In this example, we look at the use of our modified probes to detect the presence of bacterial DNA in a sample using a nanopore device.

We synthesized a probe with a PNA molecule capable of specifically binding to Staphylococcus mitis (S. mitis) bacterial DNA. The bisPNA contains a sequence complementary to a sequence that is specific for the S. mitis bacteria species.

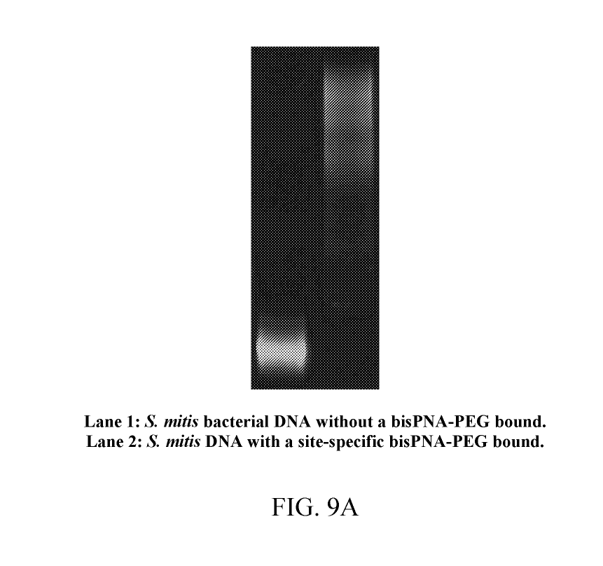

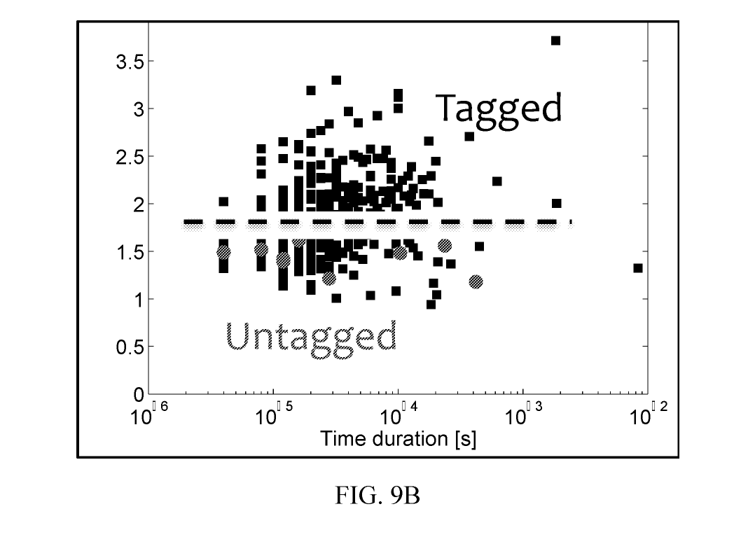

In this assay, the PNA probe is bound to 10 kDa PEG to allow for detection in a nanopore when bound to the bacterial DNA. We mixed the PNA probe with the bacterial DNA and performed a gel shift assay on the sample to observe binding. FIG. 9A shows the results of the gel shift assay, with lane 1 comprising bacterial DNA without the PNA probe, and lane 2 comprising bacterial DNA with the PNA probe. Our observed results show that our PNA/PEG (10 kDa) probe bound to the S. mitis bacterial DNA.

We next prepared two samples for detection in a nanopore. The first sample included bacterial DNA with PEG-modified PNA probes (DNA/bisPNA-PEG). The second sample included bacterial DNA alone. We ran these samples through a nanopore device in two consecutive experiments, and analyzed the resulting events. FIG. 9B shows a scatter plot of mean conductance shift (dG) on the vertical axis vs. duration on the horizontal axis for all recorded events in the two consecutive experiments. Events from tagged sample 1 (squares) and untagged sample 2 (circles) are shown.

The tagged molecules are consistently above a background threshold (dashed line), while untagged molecules are below the line and consistent with a background population. The population of molecules from a variety of background experiments (DNA/PNA without PEG, filtered serum, etc.) are used to establish the threshold (line) for flagging tagged events. Background events are not shown here. For accurate detection of bacterial DNA in a sample, the DNA must be tagged using a highly site-specific probe.

Our results show that the PNA/PEG bound population of S. mitis bacterial DNA is discernable from background events while DNA only and DNA/PNA only are not. Thus, the modified PNA-PEG sequence specific probe allows confident detection of the presence or absence of S. mitis DNA in a sample.

### Example 5—Fragmentation and Ligation for Target Sequence Detection in a Nanopore Device

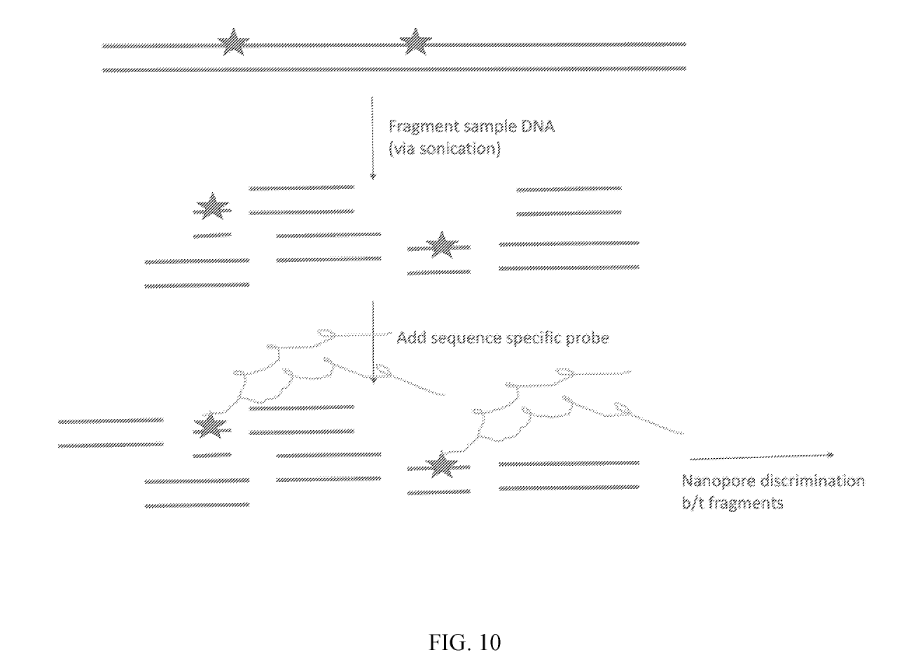

We show successful detection of the target sequence 5′-TCCCCTCCTTTT-3′ (SEQ ID NO: 1) in the plasmid genome of an E. coli bacteria as follows: Plasmid DNA from E. coli was isolated and fragmented. The fragmented DNA was exposed to site-specific probe that binds specifically to the target sequence. The probe was bound to a payload molecule to facilitate detection. Probe-payload bound polynucleotide fragments were successfully detected in a nanopore device (FIG. 10).

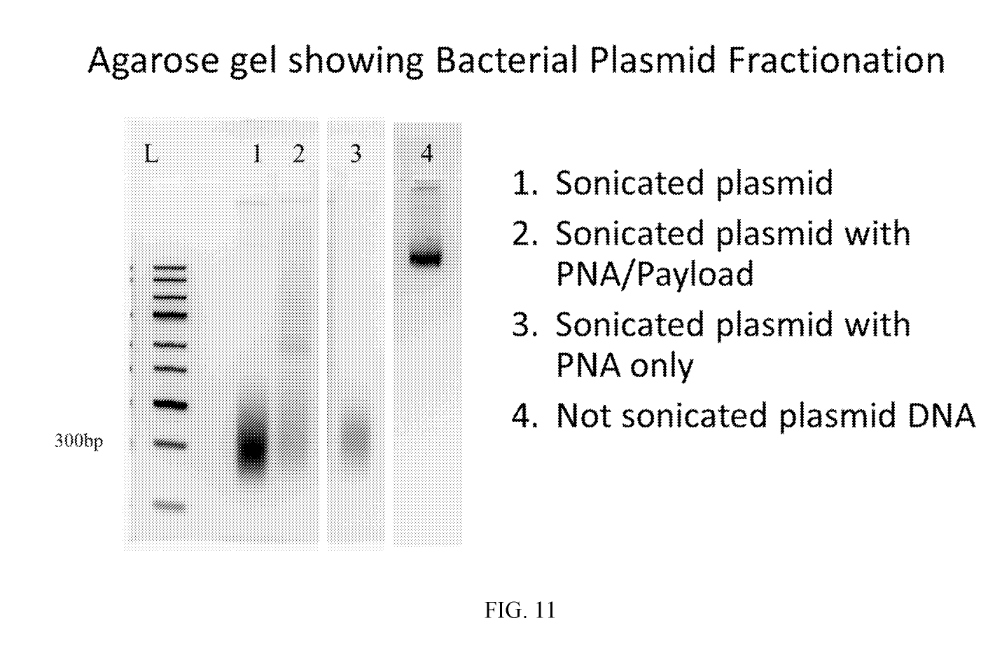

To isolate plasmid DNA from E. coli, harvested E. coli bacteria were lysed and 5.6 kbp plasmid DNA was isolated using standard silica columns and eluted in water. DNA was then fractionated to approximately 300 bp using sonication, and then incubated for 1 hr at 65° C. with the sequence specific (TCCCCTCCTTTT—SEQ ID NO: 1) bisPNA probe-payload that binds to fractionated DNA molecules that contain the perfectly matching cognate sequence. An agarose electrophoresis gel shift assay was performed to show the portion (11%) of the fractionated DNA that contains the target DNA site is bound by the probe-payload (FIG. 11).

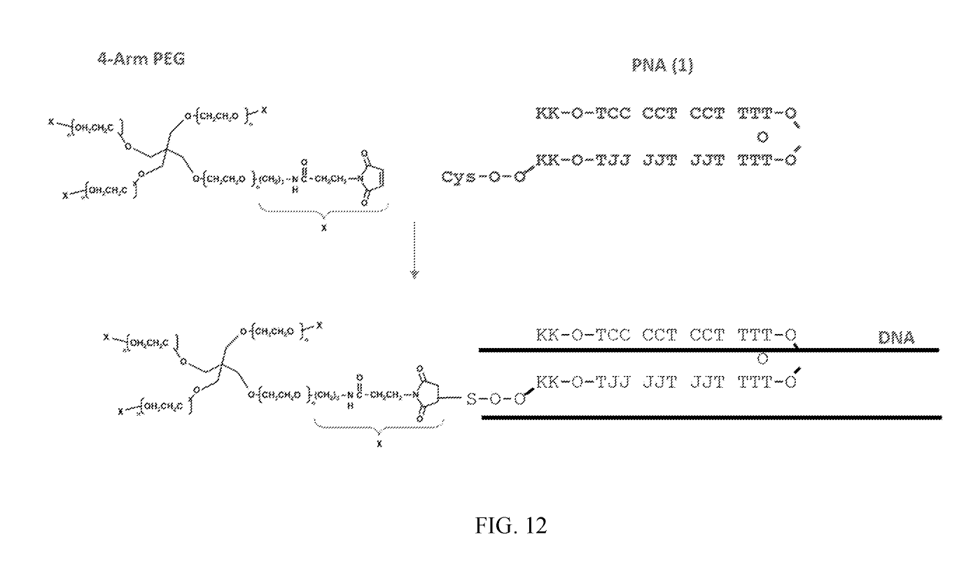

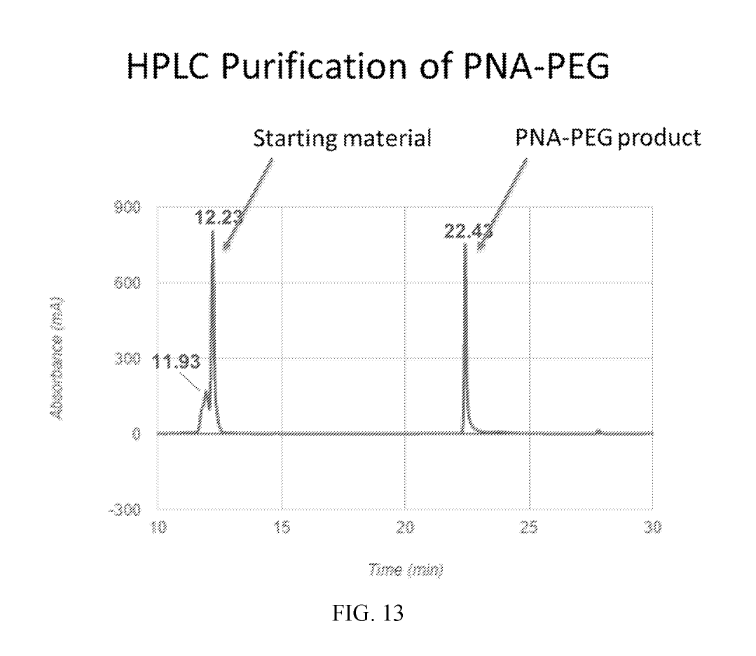

The bisPNA probe contains nucleotide bases that perfectly match the DNA sequence 5′-AAAAGGAGGGGA-3′ (SEQ ID NO: 2), N- and C-terminus Lysines (to aid in DNA binding), and a C-terminal Cysteine that allows covalent conjugation to a 40 kDa, 3-arm maleimido-PEG payload (FIG. 12). Post conjugation the bisPNA-PEG was isolated from side products and unreacted starting material using HPLC purification (FIG. 13).

The collection of fragmented DNA (probe-payload bound fragments and unbound fragments) were mixed with recording buffer (final buffer composition of 1.5 M LiCl, 10 mM NaPhosphate, 1 mM EDTA, pH 8.8), introduced to the chamber above the pore, and driven through a ˜35 nanometer nanopore using a voltage of −150 mV.

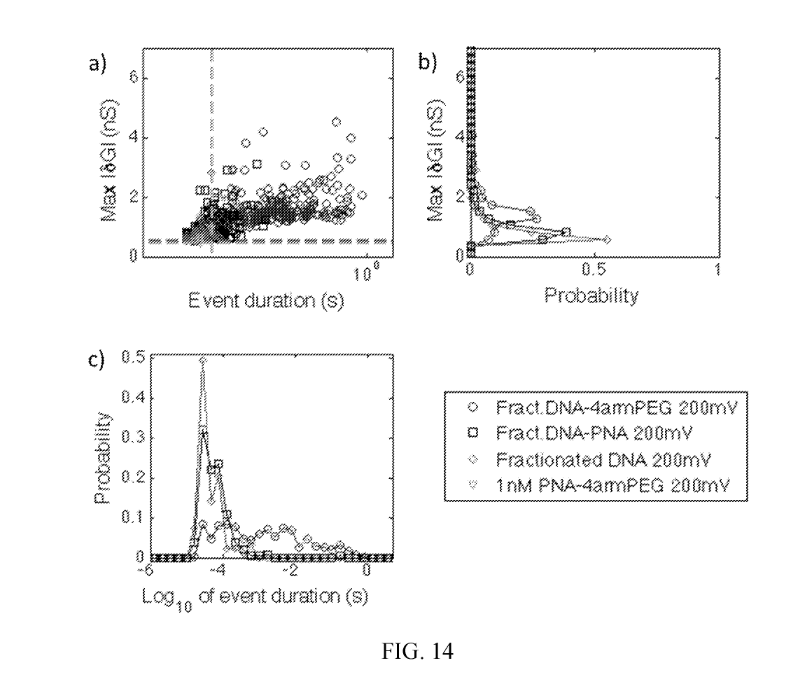

Comparing the resulting population of probe-payload exposed DNA to control samples of DNA fragments with no probes and DNA fragments with probe only (no payload), showed a new population emerged. FIG. 14, panel a) a shows a 2-D plot, with time (event duration, milliseconds) on the X-axis and current blockage (max deltaG, nano Siemens) on the Y-axis, for 4 samples. Each DNA molecule that passes through the pore is plotted based on these two criteria, the time it took to traverse through the pore and the amount of current it blocked while occluding the pore. The red population shows DNA fragments only, the black population shows DNA-Probe, the blue population shows DNA fragments that were exposed to probe-payloads, and the green are probe-payloads only (no DNA). The blue population clearly shows short DNA fragments with probe-payload attached are easily differentiated from DNA fragments without probes and DNA fragments without payload (but with probe), thus showing the importance of the probe-payload combination for detecting DNA fragments containing a target sequence of interest.

The difference in the 4 different samples is also realized when viewing the histograms for each sample. FIG. 14, panels b) and c) shows the probability for any one event to be of particular depth (dG) or duration (seconds), respectively. It can be seen that DNA and DNA/Probe provide a very similar event signature when going through the pore, while DNA/PNA-Payload is clearly differentiated. As expected, the blue sample (fragmented DNA that was exposed to the PNA-Payload) has two main populations, one that over laps with the red sample, representing the DNA fragments without PNA-Payload (of which is 89% of the population), and one that is different, representing the DNA/PNA-payload population, which is ˜11% of the total DNA fragment population.

Identifying the target sequences in this manner is may be used, for example, to identify pathogenicity, such as antibiotic resistance mutations or gene cassettes, along with other application such as monitoring horizontal gene transfer between species of bacteria, or between bacteria and viruses.

### Example 6—Quantification of Target Sequence in a Nanopore

Although the DNA/PNA-payload population from Example 5 is 11% of the total DNA fragment population, the event plots show that the DNA/PNA-payload events are significantly more than 11% of the total detected event population. In the example, from the FIG. 14, panel c) duration histogram, the payload-bound fragments are ˜70% of the total event population. This enhanced detection of the target fragment is by design via the payload probe detection mechanism, i.e., to distinguish the event signature of the target-containing fragments from all other detected events. In order to determine the concentration of the DNA/PNA-payload population, and therefore the target-containing fragment, requires a method to compensate for the enhanced detection of the target molecule. In general, it may not be possible to identify the 11:89 target:non-target ratio from the detected event ratio of the payload-bound targets vs. non-targets, since, as shown in FIG. 14, the ratio of detected event types is significantly different from the ratio of molecule types in the chamber of molecules. Thus, we introduce herein a method to quantify the target molecule in a sample using a secondary molecule type that is detectable in the nanopore with a unique event signature (either alone or bound to a probe/probe-payload complex) from the target molecule-payload complex and from non-target fragments.

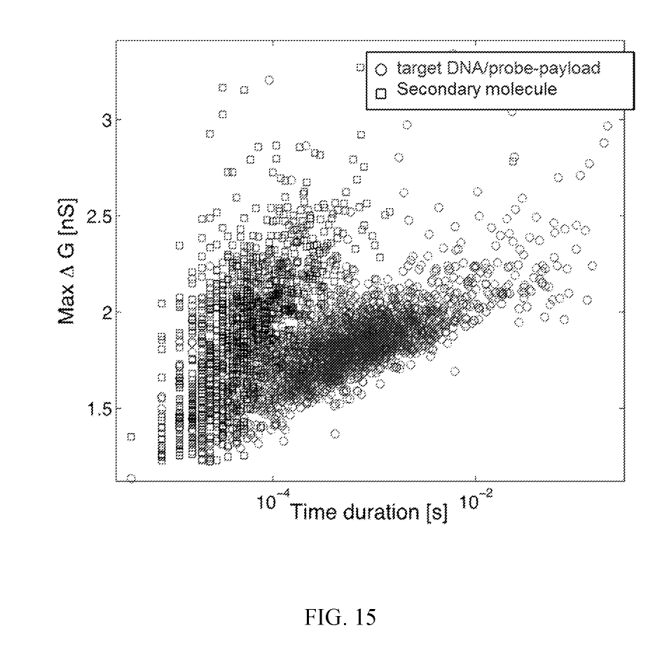

As a representative example for the fractional abundance calculation method, FIG. 15 shows an event plot for two molecule types that were run sequentially on the same pore. First, a sample containing a 96 bp DNA/probe-payload complex was prepared and measured in a nanopore device. The complex is a model for the target 300 bp fragment bound with a probe-payload in FIG. 14. The probe-payload was a PNA-PEG with a 4-arm PEG structure. Next, a sample containing secondary molecule was placed in to the nanopore device and measured. The secondary molecule was designed to generate a unique event signature upon translocation through the nanopore with which fractional abundance calculations could be achieved. The secondary molecule is a 74 bp DNA with PNA-PEG bound, where the PEG has an 8-arm structure. The secondary molecule could instead be dsDNA, e.g., 5 kb or longer. Alternatively, the secondary molecule could be a DNA of any known length with a probe-payload bound, as in this example. The key is that the secondary molecule generates a unique event subpopulation that is distinct from the target/probe-payload molecule or most other background events.

After generating the unique event signature distributions for the target DNA/probe-payload and the secondary molecule (FIG. 15), we analyzed the distributions to determine fractional abundance according to our method.

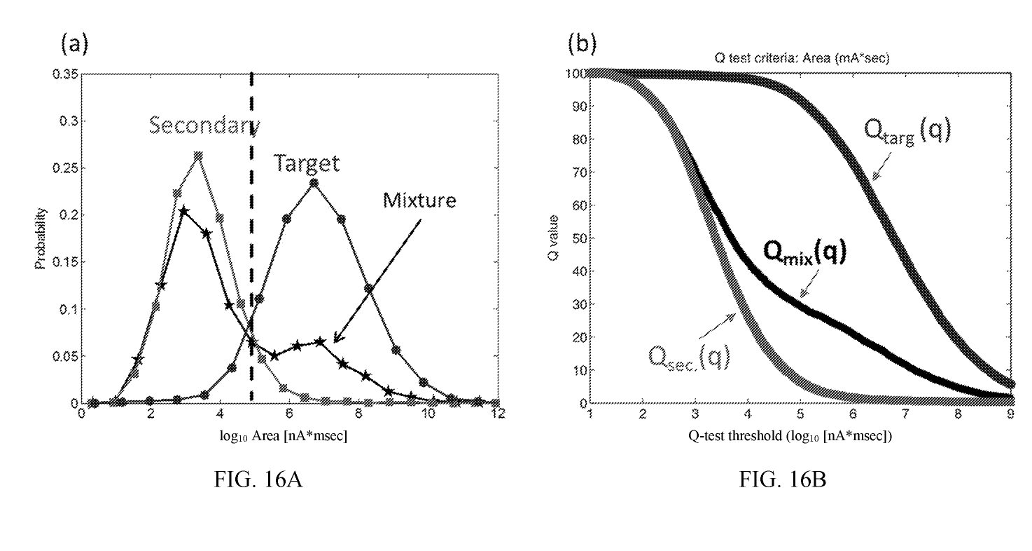

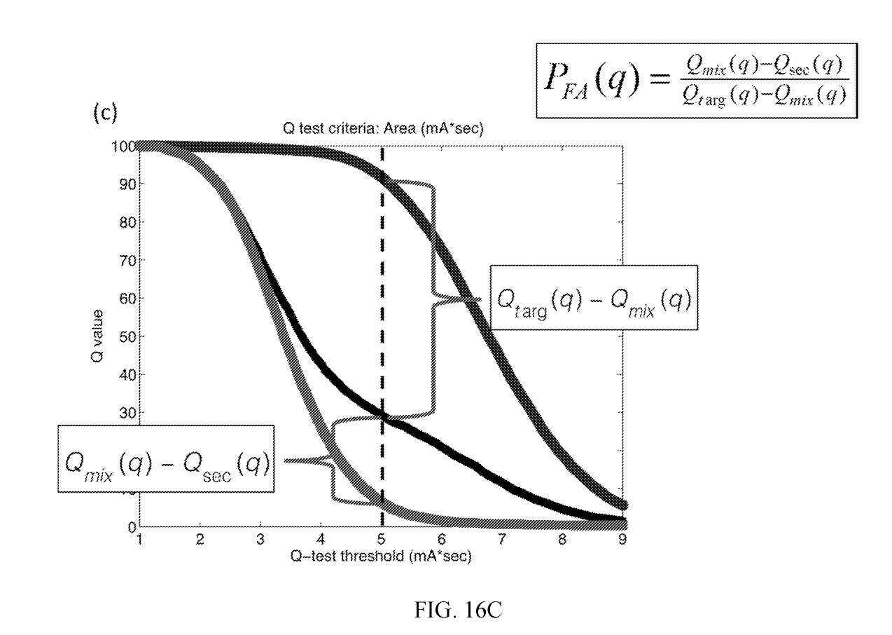

FIGS. 16A-C presents an example of how we can determine fractional abundance (FA) of the molecules in FIG. 15. Specifically, the event populations for the target-payload (or “target” for short) and secondary molecules, run individually as isolated controls, were binned into histograms using one or more event signatures such that the histogram modes are as distinct as possible. In this example, the event log₁₀ Area (where Area is defined as the average current depth (nA) of an event signature multiplied by the duration (msec) of the event signature) is used (FIG. 16A). This measure allows us to differentiate between the secondary molecules and the target-payload complexes on a linear scale to develop population histograms for further analysis. Here, the secondary molecule has a smaller area than the target-payload molecule, owing largely due to the faster duration of average event signatures for the secondary molecule (FIG. 15). We then determined Q(q)=the fraction of events with area exceeding threshold q. This was plotted for the target and the secondary molecule in FIG. 16B. A mixture of the two molecule types was also added to the nanopore device and event signatures were detected from the nanopore. From the collected data, we generated a mixed mode histogram (FIG. 16A) and corresponding Q(q) curve (FIG. 16B). In the example, the known mixture ratio we used was 3:10 target:secondary molecule, corresponding to a fractional amount of 30% for the target molecule. Note that fractional amount is defined as the fraction of concentration of the target to the secondary molecule, not as the ratio of target to total. FIG. 16C illustrates how the information provided in FIG. 16B was used to compute P_{FA}(q), defined as the predicted fractional amount of the target in the mixture. The P_{FA}(q) was computed over the q range from the 25% quintile of the secondary molecule to the 75% quintile of the target molecule, and averaged. In this example, the mean P_{FA}(q) was determined to be 0.322, which corresponds well with the true FA of 0.30.

There are other design elements to the framework that can improve performance in the context of this application. For example, we can run the isolated controls prior to and after the unknown mixture, and averaging the Q(q) values, to improve performance in some cases.

The capture rate constant (e.g., 100-200 (min*nM)−1 for the two types here) and the need for 100-200 events to achieve desired confidence suggests that total nanopore sensing time (controls, mixtures) can be done in a few minutes at nM concentrations. Also, when capture probability/rate of the two molecule types (target and secondary) are sufficiently different, bias in the estimation can be introduced. This can be compensated for by using information in the different capture rate constants for the two molecule types, as established when running the controls. Running a control mixture (e.g., 50:50) can also identify the amount of bias present and can be used to cancel it out (subtraction/inversion). This compensation can take the form of a parameter that multiplies the Qmix(q) term in the P_{FA}(q) equation.

Using the framework described, the fractional amount (defined as the ratio of target/payload molecule concentration-to-secondary molecule concentration) from FIGS. 15 and 16A-C was predicted. Table 2 shows the results. Each row in the table is a separate nanopore experiments, in which one or more mixtures were treated as unknowns and the framework was applied. Individually, prediction errors are 10% or better. By aggregating information from more than one nanopore, prediction errors can be reduced further (6% or better in the example here).

TABLE 2
Results of Fractional Abundance Determination

| True FA (%) | 10% | 15% | 20% | 25% | 30% | 40% |
|---|---|---|---|---|---|---|
| FA (%) | 12 ± 2.7% | 16.6 ± 3.1% | 19.4 ± 3.4% | 18.5 ± 3.1% | 30.6 ± 3.6% | 40.7 ± 5.4% |
| Pred.(per run) | 17.6 ± 2.3% | 17.0 ± 2.2% | 20.8 ± 2.2% | 26.1 ± 2.5% | 24.8 ± 2.3% | 50.8 ± 4.6% |
| | | | 12.1 ± 3.2% | | 24.8 ± 4.2% | |
| | | | 29.6 ± 3.3% | | 33.0 ± 3.5% | |
| Combined | 14.8 ± 1.8% | 16.8 ± 1.9% | 20.5 ± 1.5% | 22.3 ± 2.0% | 28.3 ± 1.7% | 45.8 ± 3.5% |
| FA (%) | | | | | | |
| Prediction | | | | | | |
| Error | 5% | 2% | <1% | 3% | 3% | 6% |

The workflow demonstrated in this example for quantitating the abundance of a target sequence in a population did not require any amplification, purification, concentration or buffer exchange steps. The results were obtained by mere sequential addition of reagents, such as PNA-probe-payloads and salt. This workflow is compatible with inexpensive, disposable sample prep cartridges, to allow a sample-in answer-out workflow in a miniaturized (handheld or desk top) unit.

### Other Embodiments

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A method of detecting the presence or absence of a target polynucleotide sequence suspected to be present in a sample, comprising:
a) fragmenting polynucleotides in a sample suspected of comprising a target polynucleotide comprising a target sequence;
b) contacting said sample with a probe adapted to bind specifically to a fragmented target polynucleotide comprising said target sequence under conditions that promote binding of said probe to said fragmented polynucleotide to form a polynucleotide-probe complex;
c) loading said sample into a nanopore device comprising a nanopore, a first chamber, and a second chamber, wherein said first and second chamber are in electrical and fluidic communication through said nanopore via a conducting fluid, and wherein said nanopore device further comprises a sensor configured to identify objects passing through the nanopore;
d) applying an electrical potential across said nanopore to induce translocation of said polynucleotide or polynucleotide-probe complex through said nanopore; and
e) detecting an electrical signal associated with the translocation of said polynucleotide or polynucleotide-probe complex through the nanopore.

2. The method of claim 1, further comprising analyzing said electrical signal to determine the presence or absence of said target polynucleotide in said sample.

3. The method of claim 1, further comprising comparing said electrical signal with a reference signal to determine a quantity of said target polynucleotide in said sample.

4. The method of claim 1, wherein said probe is bound to a payload molecule.

5. The method of claim 1, wherein said probe comprises a payload binding moiety.

6. The method of claim 5, wherein said payload binding moiety comprises a chemical group, a reactive group, a small molecule, or a peptide.

7. The method of claim 6, wherein the small molecule comprises biotin.

8. The method of claim 6, wherein the reactive group comprises dibenzocyclooctyl (DBCO) or azide.

9. The method of claim 6, wherein the reactive group comprises a reactive maleimide, a free thiol (thiolate), or a sulfur atom.

10. The method of claim 5, further comprising binding a payload molecule to said payload binding moiety before applying said electrical potential.

11. The method of claim 10, wherein said payload molecule is bound to said payload binding moiety after contacting said sample with said probe.

12. The method of claim 10, wherein said payload molecule is bound to said payload binding moiety before contacting said sample with said probe.

13. The method of claim 4, wherein the payload molecule is selected from the group consisting of: a dendrimer, double stranded DNA, single stranded DNA, a DNA aptamer, a fluorophore, a protein, an antibody, a polypeptide, a nanobead, a nanorod, a nanotube, nanoparticle, fullerene, a PEG molecule, a liposome, or a cholesterol-DNA hybrid.

14. The method of claim 4, wherein said payload molecule is charged.

15. The method of claim 14, wherein said charged payload molecule is selected from the group consisting of: a peptide, an amino acid, a charged nanoparticle, a synthetic molecule, a nucleotide, a polynucleotide, a metal, and an ion.

16. The method of claim 14, wherein the sensitivity or specificity of detection of the presence of absence of the target polynucleotide by said nanopore device is increased when said target polynucleotide is bound to said charged payload molecule as compared to unbound target polynucleotide.

17. The method of claim 5, wherein the payload binding moiety and the payload molecule are bound via a covalent bond.

18. The method of claim 17, wherein said covalent bond is formed by click chemistry.

19. The method of claim 18, wherein said click chemistry is copper catalyzed.

20. The method of claim 18, wherein said click chemistry is copper free.

21. The method of claim 17, wherein said covalent bond comprises a thio-ether bond.

22. The method of claim 21, wherein said thio-ether bond is formed by maleimido-thiolate chemistry.

23. The method of claim 5, wherein the payload binding moiety and the payload molecule are bound via a non-covalent bond.

24. The method of claim 23, wherein said non-covalent bond is selected from the group consisting of: a hydrogen bond, an ionic bond, a van der Waals interaction, a hydrophobic interaction, a polar bond, a cation-pi interaction, a planar stacking interaction, and a metallic bond.

25. The method of claim 4, wherein the sensitivity or specificity of detection of the presence or absence of the target polynucleotide is increased when said target polynucleotide is bound to said payload molecule as compared to unbound target polynucleotide.

26. The method of claim 4, wherein two or more payload molecules are bound to the fragmented target polynucleotide.

27. The method of claim 1, wherein said specific binding of said probe to said fragmented target polynucleotide comprising said target sequence occurs via sequence-specific ligation.

28. The method of claim 1, wherein fragmenting said polynucleotide comprises exposing said sample to a fragmentation condition.

29. The method of claim 28, wherein said fragmentation condition is selected from the group consisting of: chemical shearing, heat and divalent metal cation, acoustic shearing, sonication, hydrodynamic shearing, nebulization, needle shearing, and French pressing.

30. The method of claim 1, wherein fragmenting said polynucleotide comprises contacting said sample with a fragmentation reagent.

31. The method of claim 30, wherein said fragmentation reagent is selected from the group consisting of: a restriction enzyme, a site-directed nuclease, endonuclease, non-specific nuclease, transposase, and catalytic DNA or RNA.

32. The method of claim 1, wherein said sample comprises a plurality of target polynucleotides comprising distinct target sequences.

33. The method of claim 32, wherein contacting said sample with said probe comprises providing a plurality of unique probes adapted to specifically bind to a plurality of fragmented target polynucleotides comprising said distinct target sequences so that each of said plurality of distinct target polynucleotide-probe complexes generates a unique and detectable signal upon translocation through the nanopore.

34. The method of claim 33, wherein detecting said electrical signal comprises detecting an electrical signal associated with the translocation of at least one of said plurality of distinct target polynucleotide-probe complexes.

35. The method of claim 1, wherein said nanopore device comprises at least two nanopores, and wherein said nanopore device is configured to apply an independently-controlled voltage across each of said at least two nanopores.

36. The method of claim 35, wherein said at least two nanopores are in series.

37. The method of claim 35, further comprising capturing said polynucleotide or polynucleotide-probe complex in at least two nanopores in said device simultaneously.

38. The method of claim 1, wherein said sample is loaded into said device before said fragmentation of said polynucleotides.

39. The method of claim 1, wherein said sample is loaded into said device after said fragmentation of said polynucleotides.

40. The method of claim 1, wherein said sample is loaded into said device before said contacting of said sample with said probe.

41. The method of claim 1, wherein said sample is loaded into said device after said contacting of said sample with said probe.

42. The method of claim 1, wherein said sample is not purified.

43. The method of claim 1, wherein said sample is not purified before said fragmentation, before contacting said sample with said probe, or before said detection in said nanopore.

44. The method of claim 1, wherein said sample is loaded into said nanopore device at a dilution of at least 1:20000, 1:10000, 1:5000, 1:2000, 1:1000, 1:500, 1:200, 1:100, 1:50, 1:20, 1:10, 1:5, 1:2, 1:1.5, 1:1.2, 1:1.1 or 1:1.05.

45. The method of claim 1, wherein said sample is loaded into said nanopore device without dilution.

46. The method of claim 1, wherein said sample comprises non-target polynucleotides, fragmentation reaction reagents, and ligation reaction reagents while in said nanopore device.

47. The method of claim 1, wherein said nanopore is at least 5 nm, 10 nm, 20 nm, 20 nm, 40 nm, or 50 nm in diameter.

48. The method of claim 1, wherein said nanopore is less than 200 nm in diameter.

49. The method of claim 1, wherein fragmenting said polynucleotides comprises a sequence-specific fragmentation reaction.

50. The method of claim 49, wherein said sequence-specific fragmentation reaction comprises site-specific restriction enzymes or CRISPR-based cleavage.

51. The method of claim 1, wherein fragmenting said polynucleotides comprises a non-sequence-specific fragmentation reaction.

52. The method of claim 51, wherein said non-sequence-specific fragmentation reaction is achieved by shearing.

53. The method of claim 1, wherein said probe is contacted with said sample in the interior space of the nanopore device.

54. The method of claim 1, wherein said target polynucleotide comprises double-stranded deoxyribonucleic acid (dsDNA), single-stranded DNA (ssDNA), peptide nucleic acid (PNA), single-stranded ribonucleic acid (ssRNA), DNA/RNA hybrid, or double-stranded ribonucleic acid (dsRNA).

55. The method of claim 1, wherein the target polynucleotide is a naturally-occurring polynucleotide.

56. The method of claim 1, wherein the target polynucleotide is an artificially-synthesized polynucleotide.

57. The method of claim 1, wherein the target polynucleotide is a recombinant polynucleotide.

58. The method of claim 1, wherein the sensor comprises an electrode pair configured to generate said electrical potential across said nanopore and to detect said electrical signal.

59. The method of claim 58, wherein the electrical signal generated when the payload-bound target polynucleotide passes through the nanopore is distinguishable from the electrical signal of background molecules.

60. The method of claim 59, wherein said electrical signal is a measure of current over time, and the electrical signal is distinguishable by its mean depth, maximum depth, duration, number of depth levels, area of depth and duration, or noise level.

61. A method of quantifying a target polynucleotide sequence in a sample, comprising:
a) fragmenting polynucleotides in a sample suspected of comprising a target polynucleotide comprising a target sequence;
b) contacting said sample with a probe adapted to bind specifically to a fragmented target polynucleotide comprising said target sequence under conditions that promote binding of said probe to said fragmented polynucleotide to form a polynucleotide-probe complex;
c) loading said sample into a nanopore device comprising a nanopore, a first chamber, and a second chamber, wherein said first and second chamber are in electrical and fluidic communication through said nanopore via a conducting fluid, and wherein said nanopore device further comprises a sensor configured to identify objects passing through the nanopore;
d) applying an electrical potential across said nanopore to induce translocation of said polynucleotide or polynucleotide-probe complex through said nanopore;
e) detecting an electrical signal associated with the translocation of said polynucleotide or polynucleotide-probe complex through the nanopore; and
f) analyzing said electrical signal to quantify said target polynucleotide sequence in said sample.

62. The method of claim 61, wherein said probe is bound to a payload molecule.

63. The method of claim 61, wherein said probe comprises a payload binding moiety.

64. The method of claim 63, wherein said payload molecule is bound to said payload binding moiety after contacting said sample with said probe.

65. The method of claim 61, wherein said quantification of said target polynucleotide comprises determining a ratio of target to control events.

66. The method of claim 61, wherein said sample comprises a known concentration of target polynucleotide.

67. The method of claim 61, further comprising performing said method on another sample comprising a known concentration of a control polynucleotide, wherein the electrical signal associated with the translocation of said control polynucleotide-probe complex has average characteristics that are distinct from the electrical signal associated with the translocation of said target polynucleotide probe complex.

68. The method of claim 67, wherein the probe adapted to bind specifically to the target polynucleotide and the probe adapted to bind specifically to the control polynucleotide are distinct.

69. A kit comprising:
a device comprising a nanopore, wherein said nanopore separates an interior space of the device into two volumes, wherein the device comprises a sensor for said nanopore adapted to identify objects passing through the nanopore;
a probe adapted to bind specifically to a fragmented target polynucleotide comprising a target sequence; and
instructions for use to detect the presence or absence of said target sequence in a sample.

70. The kit of claim 69, wherein said probe is bound to a payload molecule.

71. The kit of claim 69, wherein said probe comprises a payload binding moiety.

72. The kit of claim 71, further comprising a payload molecule adapted to bind to said payload binding moiety.

73. The kit of claim 69, further comprising reagents for fragmenting said polynucleotide.
